(19) United States
(12) Patent Application Publication
(10) Pub. No.: US 2021/0122724 A1
(43) Pub. Date: Apr. 29, 2021

(54) TETRACARBOXYLIC DIANHYDRIDE, CARBONYL COMPOUND, POLYIMIDE PRECURSOR RESIN, AND POLYIMIDE

(71) Applicant: ENEOS Corporation, Tokyo (JP)

(72) Inventors: Daisuke WATANABE, Tokyo (JP); Takahiro HASEGAWA, Tokyo (JP); Asako KYOBU, Tokyo (JP)

(73) Assignee: ENEOS Corporation, Tokyo (JP)

(21) Appl. No.: 17/257,667

(22) PCT Filed: Jul. 4, 2019
(86) PCT No.: PCT/JP2019/026600

§ 371 Date: Jan. 4, 2021

(30) Foreign Application Priority Data
Jul. 9, 2018 (JP) 2018-129948

(51) Int. Cl. C07D 307/93; C07C 69/753; C08G 73/10

(52) U.S. Cl. CPC C07D 307/93; C07C 2602/42; C08G 73/10; C07C 69/753

(57) ABSTRACT

A tetracarboxylic dianhydride which is a compound represented by the following general formula (1):

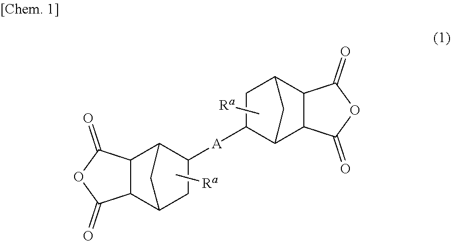

[in the formula (1), A represents one selected from the group consisting of optionally substituted divalent aromatic groups in each of which the number of carbon atoms forming an aromatic ring is 6 to 30, and R^{a}s each independently represent a hydrogen atom or the like], wherein 60% by mass or more of a stereoisomer contained in the compound is an exo/exo type stereoisomer represented by a specific general formula.

### TECHNICAL FIELD

The present invention relates to a tetracarboxylic dianhydride, a carbonyl compound, a polyimide precursor resin, and a polyimide.

### BACKGROUND ART

In the field of display equipment such as displays that use organic electroluminescence elements and liquid crystal displays, as a material used for substrates and the like, the advent of a material having high light transmittance and sufficiently high heat resistance such as glass has been required. In recent years, polyimides have attracted attention as a material used as a substitute for glass, and various tetracarboxylic dianhydrides have been studied as monomers for producing such polyimides.

For example, International Publication No. WO2015/163314 (PTL 1) and Japanese Unexamined Patent Application Publication No. 2018-44180 (PTL 2) disclose a tetracarboxylic dianhydride represented by the following formula (a):

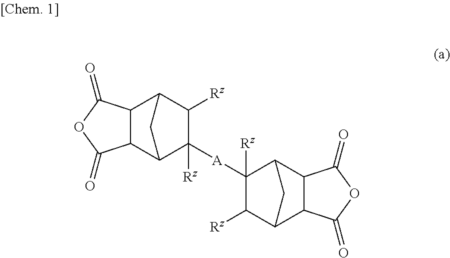

[in the formula (a), A represents one selected from the group consisting of optionally substituted divalent aromatic groups in each of which the number of carbon atoms forming an aromatic ring is 6 to 30, and multiple R^{z}s each independently represent one selected from the group consisting of a hydrogen atom and alkyl groups having 1 to 10 carbon atoms]. Note that Synthesis Example 2 of PTL 2 synthesizes a compound in which, in the above formula, A is a benzene ring and each of the R^{z}s is a hydrogen atom, and the three-dimensional structure of that compound has a structure in which each acid anhydride group has an endo conformation with respect to the norbornane ring to be bonded. What is actually demonstrated in the Synthetic Example is made up of an endo/endo type stereoisomer.

Note that, in PTL 1, examples of the raw material of the tetracarboxylic dianhydride represented by the above formula (a) include nadic anhydride, 5-methylnadic anhydride, 5,6-dimethylnadic anhydride, 5-ethyl-6-methylnadic anhydride, 5,6-diethylnadic anhydride, 5-methyl-6-isopropylnadic anhydride, 5-n-butylnadic anhydride, and the like, and Examples use 5-norbornene-2,3-dicarboxylic anhydride. In addition, PTL 2 also uses 5-norbornene-2,3-dicarboxylic anhydride in Synthesis Example 2 thereof as a raw material for the tetracarboxylic dianhydride represented by the above formula (a). Such 5-norbornene-2,3-dicarboxylic anhydride (nadic anhydride) is generally produced by utilizing the Diels-Alder reaction between cyclopentadiene and maleic anhydride. In the Diels-Alder reaction, the endo adduct is a kinetically advantageous product and is preferentially produced over the exo adduct (endo rule). Therefore, in the case of employing a general method for producing nadic anhydride, an endo form is formed basically (one having a structure in which an acid dianhydride bonded to the norbornane ring is bonded to the norbornane ring in an endo configuration). Here, Synthesis Example 2 of above PTL 2 produces a tetracarboxylic dianhydride represented by the above formula (a) using 5-norbornene-2,3-dicarboxylic anhydride (nadic anhydride) without specifying the configuration such as endo or exo, and as described above, the obtained tetracarboxylic dianhydride is made up of the endo/endo type stereoisomer in which each acid anhydride group has an endo conformation with respect to the norbornane ring to be bonded.

### CITATION LIST

### Patent Literature

[PTL 1] International Publication No. WO2015/163314

[PTL 2] Japanese Unexamined Patent Application Publication No. 2018-44180

### SUMMARY OF INVENTION

### Technical Problem

The tetracarboxylic dianhydride represented by the above formula (a) described in PTLs 1 and 2 has high light transmittance and sufficiently high heat resistance when polyimide is produced using such a compound as a monomer. However, the tetracarboxylic dianhydride represented by the above formula (a) described in above PTLs 1 and 2 is not necessarily sufficient in that the linear expansion coefficient is set to a lower value when polyimide is produced using such a compound as a monomer.

The present invention has been made in view of the problems of the related art, and an object thereof is to provide a tetracarboxylic dianhydride that can be used as a raw material monomer for producing a polyimide having a lower linear expansion coefficient while having a sufficiently high level of light transmittance and heat resistance; a carbonyl compound that can be used as a raw material for efficiently producing the tetracarboxylic dianhydride and can be obtained as an intermediate during the production of the tetracarboxylic dianhydride; a polyimide precursor resin that can be suitably used for producing the polyimide having a lower linear expansion coefficient while having a sufficiently high level of light transmittance and heat resistance and can be efficiently produced by using the tetracarboxylic dianhydride; and a polyimide that can have a lower linear expansion coefficient while having a sufficiently high level of light transmittance and heat resistance.

### Solution to Problem

Regarding the conventional 5-norbornene-2,3-dicarboxylic anhydride (nadic anhydride) used for producing a tetracarboxylic di anhydride represented by the above formula (a) described in above PTLs 1 and 2, all of those having no explicit configuration such as endo and exo contain 97% by mass or more of the endo form (endo-nadic anhydride). Therefore, the conventional tetracarboxylic dianhydride represented by the above formula (a) has, as described in Synthesis Example 2 of above PTL 2, a structure in which each acid anhydride group has an endo conformation with respect to the norbornane ring to be bonded. Meanwhile, the present inventors made earnest studies to achieve the above object, and found as a result that, in the compound represented by the following general formula (1) (tetracarboxylic dianhydride), when 60% by mass or more of a stereoisomer contained in the compound is an exo/exo type stereoisomer represented by the following general formula (2), it is possible to produce a polyimide having a lower linear expansion coefficient while having a sufficiently high level of light transmittance and heat resistance in the case of forming a polyimide using such a compound (tetracarboxylic dianhydride). Thus, the present invention has been completed.

Specifically, a tetracarboxylic dianhydride of the present invention is a compound represented by the following general formula (1):

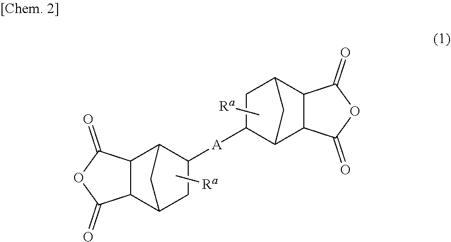

[in the formula (1), A represents one selected from the group consisting of optionally substituted divalent aromatic groups in each of which the number of carbon atoms forming an aromatic ring is 6 to 30, and R^{a}s each independently represent one selected from the group consisting of a hydrogen atom and alkyl groups having 1 to 10 carbon atoms], wherein 60% by mass or more of a stereoisomer contained in the compound is an exo/exo type stereoisomer represented by the following general formula (2):

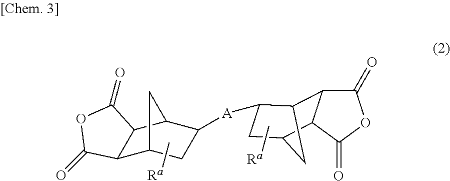

[A and R^{a} in the formula (2) have the same definitions as A and R^{a} in the above general formula (1)]. Note that, regarding the stereoisomers of the compound represented by the general formula (1), the “exo/exo type” indicates that any acid anhydride group bonded to the norbornane ring in the compound has an exo conformation with respect to the norbornane ring to be bonded, that is, each acid anhydride group is present at the exo position with respect to the norbornane ring to be bonded (each acid anhydride group has an exo conformation).

In addition, a carbonyl compound of the present invention is a compound represented by the following general formula (3):

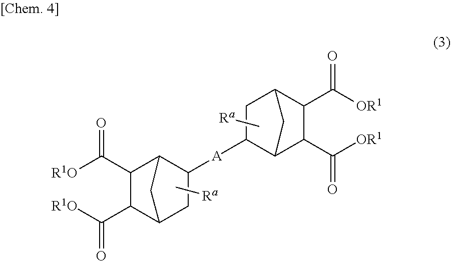

[in the formula (3), A represents one selected from the group consisting of optionally substituted divalent aromatic groups in each of which the number of carbon atoms forming an aromatic ring is 6 to 30, R^{a}s each independently represent one selected from the group consisting of a hydrogen atom and alkyl groups having 1 to 10 carbon atoms, and R¹s each independently represent one selected from the group consisting of a hydrogen atom, alkyl groups having 1 to 10 carbon atoms, cycloalkyl groups having 3 to 10 carbon atoms, alkenyl groups having 2 to 10 carbon atoms, aryl groups having 6 to 20 carbon atoms, and aralkyl groups having 7 to 20 carbon atoms], wherein 60% by mass or more of a stereoisomer contained in the compound is an exo/exo type stereoisomer represented by the following general formula (4):

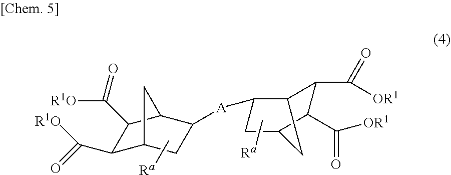

[A, R^{a}, and R¹ in the formula (4) have the same definitions as A, R^{a}, and R¹ in the above general formula (3), respectively]. Note that, regarding the stereoisomers of the compound represented by the general formula (3), the “exo/exo type” indicates that any ester group(group represented by —COOR¹) bonded to the norbornane ring in the compound has an exo conformation with respect to the norbornane ring to which the group is bonded, that is, each ester group (group represented by —COOR¹) is present at the exo position with respect to the norbornane ring to be bonded (each acid anhydride group has an exo conformation).

In addition, a polyimide precursor resin of the present invention is a polyimide precursor resin comprising a repeating unit (I) represented by the following general formula (5):

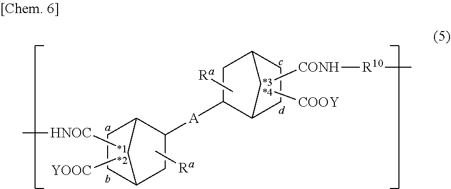

[in the formula (5), A represents one selected from the group consisting of optionally substituted divalent aromatic groups in each of which the number of carbon atoms forming an aromatic ring is 6 to 30, R^{a}s each independently represent one selected from the group consisting of a hydrogen atom and alkyl groups having 1 to 10 carbon atoms, R¹⁰ represents an arylene group having 6 to 50 carbon atoms, Ys each independently represent one selected from the group consisting of a hydrogen atom, alkyl groups having 1 to 6 carbon atoms, and alkylsilyl groups having 3 to 9 carbon atoms, one of the bonder represented by *1 and the bonder represented by *2 is bonded to the carbon atom a forming the norbornane ring, the other of the bonder represented by *1 and the bonder represented by *2 is bonded to the carbon atom b forming the norbornane ring, one of the bonder represented by *3 and the bonder represented by *4 is bonded to the carbon atom c forming the norbornane ring, and the other of the bonder represented by *3 and the bonder represented by *4 is bonded to the carbon atom d forming the norbornane ring], wherein

60% by mass or more of the repeating unit (I) contained in the polyimide precursor resin is a repeating unit having an exo/exo type three-dimensional structure represented by the following general formula (6):

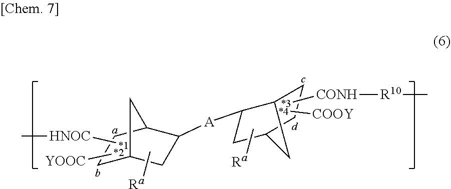

[in the formula (6), A, R^{a}, R¹⁰, and Y have the same definitions as A, R^{a}, R¹⁰, and Y in the general formula (5), respectively, one of the bonder represented by *1 and the bonder represented by *2 is bonded to the carbon atom a forming the norbornane ring, the other of the bonder represented by *1 and the bonder represented by *2 is bonded to the carbon atom b forming the norbornane ring, one of the bonder represented by *3 and the bonder represented by *4 is bonded to the carbon atom c forming the norbornane ring, the other of the bonder represented by *3 and the bonder represented by *4 is bonded to the carbon atom d forming the norbornane ring, and the bonders represented by *1 to *4 have an exo conformation with respect to the norbornane ring to be bonded]. Note that, regarding the repeating unit (I), the “exo/exo type three-dimensional structure” refers to a three-dimensional structure in which the bonders represented by *1 to *4 each take an exo conformation with respect to the norbornane ring to be bonded.

Moreover, a polyimide of the present invention is a polyimide comprising a repeating unit (A) represented by the following general formula (7):

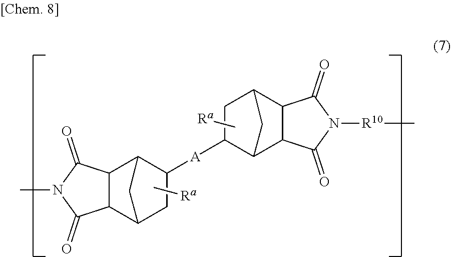

[in the formula (7), A represents one selected from the group consisting of optionally substituted divalent aromatic groups in each of which the number of carbon atoms forming an aromatic ring is 6 to 30, R^{a}s each independently represent one selected from the group consisting of a hydrogen atom and alkyl groups having 1 to 10 carbon atoms, and R¹⁰ represents an arylene group having 6 to 50 carbon atoms], wherein

60% by mass or more of the repeating unit (A) contained in the polyimide is a repeating unit having an exo/exo type three-dimensional structure represented by the following general formula (8):

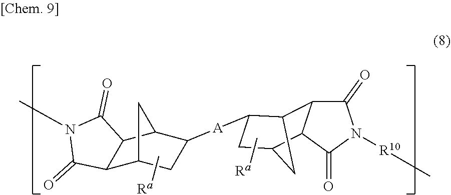

[A, R^{a}, and R¹⁰ in the formula (8) have the same definitions as A, R^{a}, and R¹⁰ in the above general formula (7), respectively]. Note that, regarding the repeating unit (A), the “exo/exo type three-dimensional structure” indicates that any imide ring bonded to the norbornane ring in the repeating unit has an exo conformation with respect to the norbornane ring to be bonded, that is, each imide ring is present at the exo position with respect to the norbornane ring to be bonded (each imide ring has an exo conformation).

### ADVANTAGEOUS EFFECTS OF INVENTION

The present invention makes it possible to provide a tetracarboxylic dianhydride that can be used as a raw material monomer for producing a polyimide having a lower linear expansion coefficient while having a sufficiently high level of light transmittance and heat resistance; a carbonyl compound that can be used as a raw material for efficiently producing the tetracarboxylicdianhydride and can be obtained as an intermediate during the production of the tetracarboxylic dianhydride; a polyimide precursor resin that can be suitably used for producing the polyimide having a lower linear expansion coefficient while having a sufficiently high level of light transmittance and heat resistance and can be efficiently produced by using the tetracarboxylic dianhydride; and a polyimide that can have a lower linear expansion coefficient while having a sufficiently high level of light transmittance and heat resistance.

### DESCRIPTION OF EMBODIMENTS

Hereinafter, the present invention is described in detail according to the preferred embodiment thereof.

### [Tetracarboxylic Dianhydride]

The tetracarboxylic dianhydride of the present invention is a compound represented by the above general formula (1), wherein 60% by mass or more of a stereoisomer contained in the compound is an exo/exo type stereoisomer represented by the above general formula (2).

A in the general formulas (1) and (2) is each an optionally substituted divalent aromatic group, and the number of carbon atoms forming an aromatic ring contained in the aromatic group is 6 to 30 (note that, in a case where the aromatic group has a substituent (such as a hydrocarbon group) containing a carbon atom(s), “the number of carbon atoms forming an aromatic ring” herein does not include the number of carbon atoms in the substituent, but refers to only the number of carbon atoms of the aromatic ring in the aromatic group. For example, in the case of a 2-ethyl-1,4-phenylene group, the number of carbon atoms forming the aromatic ring is 6). As described above, A in the above general formulas (1) and (2) is an optionally substituted divalent group (divalent aromatic group) having an aromatic ring having 6 to 30 carbon atoms. If the number of carbon atoms forming an aromatic ring exceeds the upper limit, a polyimide tends to be colored in the case of forming the polyimide using the tetracarboxylic dianhydride as a raw material. In addition, from the viewpoints of transparency and ease of purification, the number of carbon atoms forming the aromatic ring of the divalent aromatic group is more preferably 6 to 18, and further preferably 6 to 12.

In addition, such A in the general formulas (1) and (2) (the divalent aromatic groups) are not particularly limited, as long as the above-described condition of the number of carbon atoms is satisfied. For example, it is possible to use, as appropriate, residues formed when two hydrogen atoms are eliminated from aromatic compounds such as benzene, naphthalene, terphenyl, anthracene, phenanthrene, triphenylene, pyrene, chrysene, biphenyl, terphenyl, quaterphenyl, and quinquephenyl (note that, regarding these residues, the positions at which the hydrogen atoms are eliminated are not particularly limited, and examples thereof include a 1,4-phenylene group, a 2,6-naphthylene group, a 2,7-naphthylene group, a 4,4′-biphenylene group, a 9,10-anthracenylene group, and the like); and groups formed when at least one hydrogen atom is replaced with a substituent in the above-described residues (for example, a 2,5-dimethyl-1,4-phenylene group and a 2,3,5,6-tetramethyl-1,4-phenylene group), and the like. Note that, in these residues, the positions at which the hydrogen atoms are eliminated are not particularly limited as described above, and, for example, when the residue is a phenylene group, the positions may be any of ortho-positions, meta-positions, and para-positions.

From the viewpoint that a better heat resistance can be obtained, such A in the general formulas (1) and (2) (the divalent aromatic groups) are preferably phenylene groups, biphenylene groups, naphthylene groups, anthracenylene groups, and terphenylene groups, each of which is optionally substituted, more preferably phenylene groups, biphenylene groups, naphthylene groups, and terphenylene groups, each of which is optionally substituted, and further preferably phenylene groups, biphenylene groups, and naphthylene groups, each of which is optionally substituted.

In addition, in A in the general formulas (1) and (2), the substituents which may be present on the divalent aromatic groups are not particularly limited, and examples thereof include alkyl groups, alkoxy groups, halogen atoms, and the like. Of these substituents which may be present on the divalent aromatic groups, alkyl groups having 1 to 10 carbon atoms and alkoxy groups having 1 to 10 carbon atoms are more preferable, from the viewpoint that the polyimide has better solubility in solvent and offers a higher processability. If the number of carbon atoms of each of the alkyl groups and the alkoxy group preferable as the substituents exceeds 10, the heat resistance of the polyimide tends to be lowered. In addition, the number of carbon atoms of each of the alkyl groups and the alkoxy groups preferable as the substituents is preferably 1 to 6, more preferably 1 to 5, further preferably 1 to 4, and particularly preferably 1 to 3, from the viewpoint that a higher heat resistance can be obtained when a polyimide is produced. In addition, each of the alkyl groups and the alkoxy groups which may be selected as the substituents may be linear or branched.

In addition, the conformation of A in the general formula (2) is not particularly limited, but from the viewpoint that the exo/exo type stereoisomer represented by the general formula (2) has a higher solubility in solvent, A preferably has an exo conformation with respect to both norbornane rings to be bonded.

Meanwhile, the alkyl group which may be selected as R^{a} in the general formulas (1) and (2) is an alkyl group having 1 to 10 carbon atoms. If the number of carbon atoms exceeds 10, the heat resistance of a polyimide obtained in the use as a monomer for the polyimide is lowered. In addition, the number of carbon atoms of the alkyl group which may be selected as R^{a} is preferably 1 to 6, more preferably 1 to 5, further preferably 1 to 4, and particularly preferably 1 to 3, from the viewpoint that a higher heat resistance can be obtained when a polyimide is produced. In addition, the alkyl group which may be selected as R^{a} may be linear or branched.

R^{a}s in the general formulas (1) and (2) are each independently more preferably a hydrogen atom, a methyl group, an ethyl group, a n-propyl group, or an isopropyl group, and particularly preferably a hydrogen atom or a methyl group, for example, from the viewpoints that a higher heat resistance can be obtained when a polyimide is produced, that the raw material is readily available, and that the purification is easier. In addition, multiple R^{a}s in the formula may be the same as one another or different from one another, and are preferably the same from the viewpoints of ease of purification and the like.

In addition, a tetracarboxylic dianhydride of the present invention is a compound represented by the above general formula (1), wherein 60% by mass or more of the stereoisomer contained in the compound is the exo/exo type stereoisomer represented by the above general formula (2). Here, in addition to the exo/exo type stereoisomers, such a compound represented by the general formula (1) may contain, as its stereoisomer, an endo/endo type stereoisomer represented by the following general formula (2′):

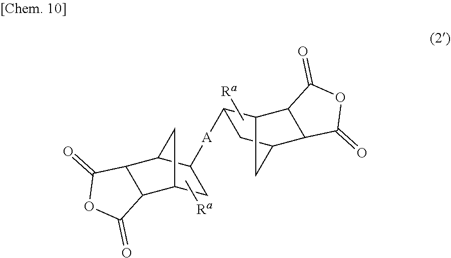

[A and R^{a} in the formula (2′) have the same definitions as A and R^{a}in the above general formula (1)]. Note that, regarding the stereoisomers of the compound represented by the general formula (1), the “endo/endo type” means that any acid dianhydride group bonded to the norbornane ring in the compound has an endo conformation with respect to the norbornane ring to be bonded.

As described above, the compound represented by the above general formula (1) may contain multiple kinds of stereoisomers, and the tetracarboxylic dianhydride of the present invention is such a compound represented by the general formula (1), wherein the content of the exo/exo type stereoisomer (the structure represented by the above general formula (2)) is 60% by mass or more. If the content of such an exo/exo type stereoisomer is less than the lower limit, when a polyimide is formed by using this as a monomer for polyimide, the linear expansion coefficient cannot be set to a lower value, and the solubility of the compound in a solvent becomes low. In addition, from the viewpoint that the linear expansion coefficient of the obtained polyimide can be set to an even lower value when used as a monomer for polyimide, the content of such an exo/exo type stereoisomer is more preferably 70% by mass or more (more preferably 80% by mass or more, and particularly preferably 90% by mass or more).

In addition, if the compound represented by the above general formula (1) contains a different stereoisomer other than the exo/exo type stereoisomer, such a different stereoisomer is preferably an endo/endo type stereoisomer.

Note that the three-dimensional structure of each stereoisomer in the compound represented by the above general formula (1) can be specified, for example, by measuring one-dimensional NMR (¹H and ¹³C) and two-dimensional NMR (DEPT 135, DQF COSY, HMQC, HMBC, NOESY). In addition, the content ratio of each stereoisomer in the compound represented by the above general formula (1) can be calculated using, for example, ¹H-NMR. The peak assigned to the proton at the bridgehead of the norbornane moiety has a chemical shift value that differs depending on each stereoisomer in the compound represented by the above general formula (1), and thus the content ratio of each stereoisomer can be obtained by taking the integration ratio of each peak.

In addition, the method for producing such a tetracarboxylic dianhydride is not particularly limited, and it is possible to employ, for example, a method similar to the method described in paragraph [0077] to paragraph [0105] of International Publication No. WO2015/163314 except that the acid anhydride as the raw material is an acid anhydride represented by the following general formula (11), wherein 60% by mass or more of the stereoisomers contained in the acid anhydride is an exo form represented by the following general formula (12) (the acid anhydride group has an exo conformation with respect to the norbornene ring) (hereinafter sometimes referred to as the “raw material compound (I)”); a method similar to the method described in paragraph [0106] to paragraph [0154] of International Publication No. WO2015/163314 except that the ester compound as the raw material is an ester compound represented by the following general formula (13), wherein 60% by mass or more of the stereoisomers contained in the ester compound is an exo form represented by the following general formula (14) in which all the ester groups bonded to the norbornene ring have an exo conformation with respect to the norbornene ring (hereinafter sometimes referred to as the “raw material compound (II)”); and the like

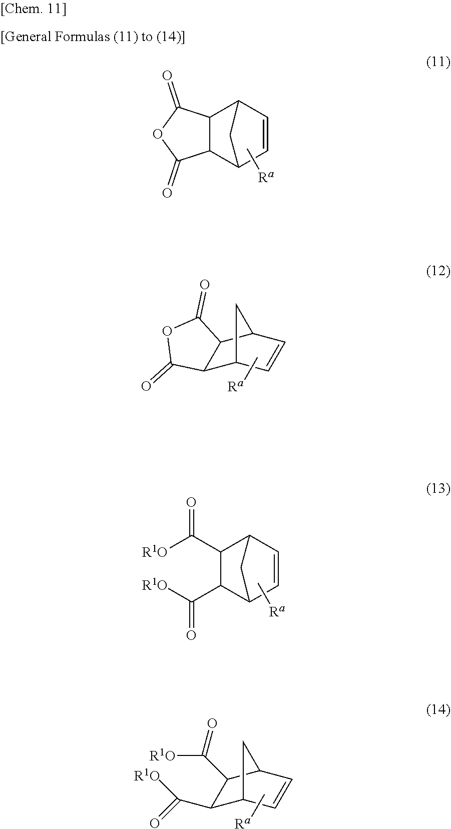

[R^{a}s in the formulas (11) to (14) have the same definitions as R^{a}s in the above general formulas (1) and (2), and R¹s in the formulas (13) to (14) have the same definitions as R′s in the general formulas (3) and (4) (note that a suitable R¹ is described together with the description of the later-described carbonyl compound)].

The method for producing the raw material compound

(I) is not particularly limited either, and a known method can be appropriately used, and a commercially available product maybe used. In addition, the ester compound (raw material compound (II)) represented by the above general formula (13) containing 60% by mass or more of the exo form represented by the above general formula (14) as a stereoisomer can be easily prepared by esterifying the raw material compound (I) with an alcohol represented by the formula: R¹OH (R¹ has the same definition as R¹ in the above general formulas (3) and (4)).

### [Carbonyl Compound]

The carbonyl compound of the present invention is a compound represented by the above general formula (3), wherein 60% by mass or more of a stereoisomer contained in the compound is an exo/exo type stereoisomer represented by above general formula (4).

Such A and R^{a}s in the general formula (3) and general formula (4) have the same definition as A and R^{a}s in the general formulas (1) and (2), respectively (the suitable ones and suitable conditions (conditions for conformation of A and the like) are also the same).

R¹s in the above general formula (3) and general formula (4) each independently represent one selected from the group consisting of a hydrogen atom, alkyl groups having 1 to 10 carbon atoms, cycloalkyl groups having 3 to 10 carbon atoms, alkenyl groups having 2 to 10 carbon atoms, aryl groups having 6 to 20 carbon atoms, and aralkyl groups having 7 to 20 carbon atoms. As described above, the alkyl group that can be selected as R¹ in the general formula (3) and general formula (4) is an alkyl group having 1 to 10 carbon atoms. If the number of carbon atoms of such an alkyl group exceeds 10, purification becomes difficult. In addition, the number of carbon atoms of the alkyl group that can be selected as the multiple R¹s is more preferably 1 to 5, and further preferably 1 to 3, from the viewpoint of facilitating purification. In addition, the alkyl group that can be selected as the multiple R¹s may be linear or branched.

In addition, the cycloalkyl group that can be selected as R¹ in the general formula (3) and general formula (4) is a cycloalkyl group having 3 to 10 carbon atoms. If the number of carbon atoms of such a cycloalkyl group exceeds 10, purification becomes difficult. In addition, the number of carbon atoms of the cycloalkyl group that can be selected as the multiple R¹s is more preferably 3 to 8, and further preferably 5 to 6, from the viewpoint of facilitating purification.

Moreover, the alkenyl group that can be selected as R¹ in the general formula (3) and general formula (4) is an alkenyl group having 2 to 10 carbon atoms. If the number of carbon atoms of such an alkenyl group exceeds 10, purification becomes difficult. In addition, the number of carbon atoms of the alkenyl group that can be selected as the multiple R¹s is more preferably 2 to 5, and further preferably 2 to 3, from the viewpoint of facilitating purification.

In addition, the aryl group that can be selected as R¹ in the general formula (3) and general formula (4) is an aryl group having 6 to 20 carbon atoms. If the number of carbon atoms in such an aryl group exceeds 20, purification becomes difficult. In addition, the number of carbon atoms of the aryl group that can be selected as the multiple R¹s is more preferably 6 to 10, and further preferably 6 to 8, from the viewpoint of facilitating purification.

In addition, the aralkyl group that can be selected as R¹ in the general formula (3) and general formula (4) is an aralkyl group having 7 to 20 carbon atoms. If the number of carbon atoms in such an aralkyl group exceeds 20, purification becomes difficult. In addition, the number of carbon atoms of the aralkyl group that can be selected as the multiple R¹s is more preferably 7 to 10, and further preferably 7 to 9, from the viewpoint of facilitating purification.

Moreover, from the viewpoint of facilitating purification, R¹ in the general formula (3) and general formula (4) is preferably an alkyl group having 1 to 5 carbon atoms, further preferably a methyl group or an ethyl group, and particularly preferably a methyl group. Note that the multiple R¹s in the general formula (3) may be the same or different, but are more preferably the same from the viewpoint of synthesis.

In addition, the carbonyl compound of the present invention is a compound represented by the above general formula (3), wherein 60% by mass or more of the stereoisomer contained in the compound is the exo/exo type stereoisomer represented by the above general formula (4). Here, in addition to the exo/exo type stereoisomers, such a compound represented by the general formula (3) may contain, as its stereoisomer, an endo/endo type stereoisomer represented by the following general formula (4′):

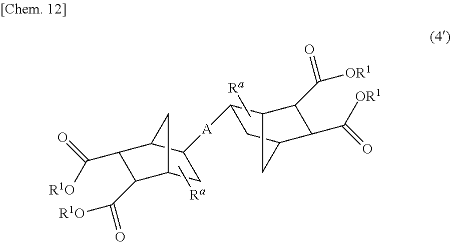

[A and R^{a} in the formula (4′) have the same definitions as A and R^{a} in the above general formula (1)]. Note that, regarding the stereoisomers of the compound represented by the general formula (3), the “endo/endo type” means that any ester group (group represented by —COOR¹) bonded to the norbornane ring in the compound has an endo conformation with respect to the norbornane ring to which the group is bonded. In addition, such an endo/endo type stereoisomer may be prepared by reacting the endo/endo type tetracarboxylic dianhydride represented by the above general formula (2′) with an alcohol (or water) represented by the formula: R¹OH [R¹ has the same definition as R¹ in the general formula (3) and general formula (4)].

As described above, the compound represented by the above general formula (3) may contain multiple kinds of stereoisomers, and the carbonyl compound of the present invention is a compound represented by the above general formula (3), wherein the content of the exo/exo type stereoisomer (the structure represented by the above general formula (4)) is 60% by mass or more. If the content of such an exo/exo type stereoisomer is less than the lower limit, the solubility of the obtained acid dianhydride in an organic solvent is reduced when the acid dianhydride is induced, and in addition, the linear expansion coefficient of the obtained polyimide cannot be set to a lower value when the acid dianhydride is used as a monomer for polyimide. In addition, from the viewpoint that the linear expansion coefficient of the obtained polyimide can be set to an even lower value when an acid dianhydride is induced and the acid dianhydride is used as a monomer for polyimide, the content of such an exo/exo type stereoisomer is more preferably 70% by mass or more (more preferably 80% by mass or more, and particularly preferably 90% by mass or more).

In addition, when the compound represented by the above general formula (3) contains different stereoisomers other than the exo/exo type stereoisomer, such different stereoisomers are preferably endo/endo type stereoisomers.

Note that the three-dimensional structure of each stereoisomer in the compound represented by the above general formula (3) can be specified, for example, by measuring one-dimensional NMR (¹H and ¹³C) and two-dimensional NMR (DEPT 135, DQF COSY, HMQC, HMBC, NOESY). In addition, the content ratio of each stereoisomer in the compound represented by the above general formula (1) can be calculated by, for example, ¹H-NMR. The peak assigned to the proton bonded to the same carbon as the ester group has a chemical shift value that differs depending on each stereoisomer in the compound represented by the above general formula (3). Therefore, the content ratio of each stereoisomer can be obtained by taking the integration ratio of each peak.

The method for producing such a carbonyl compound is not particularly limited, and it is possible to employ, for example, a method of reacting the above tetracarboxylic dianhydride of the present invention with an alcohol represented by the formula: R¹OH [R¹ has the same definition as R¹ in the general formula (3) and general formula (4)], or it is possible to employ a production method using a step similar to the step (A) described in paragraph [0106] to paragraph [0138] of International Publication No. WO2015/163314 except that the raw material compound (II) is used as the raw material ester compound.

### [Polyimide Precursor Resin]

The polyimide precursor resin of the present invention is a polyimide precursor resin comprising a repeating unit (I) represented by the above general formula (5), wherein 60% by mass or more of the repeating unit (I) contained in the polyimide precursor resin is a repeating unit having an exo/exo type three-dimensional structure represented by the above general formula (6).

Such A and R^{a}s in the general formula (5) and general formula (6) have the same definition as A and R^{a}s in the general formulas (1) and (2), respectively (the suitable ones and suitable conditions (conditions for conformation of A and the like) are also the same).

In addition, the arylene group that can be selected as R¹⁰ in the general formulas (5) and (6) is an arylene group having 6 to 50 carbon atoms. The number of carbon atoms of such an arylene group is preferably 6 to 40, more preferably 6 to 30, and further preferably 12 to 20. If the number of carbon atoms is less than the lower limit, the heat resistance of the polyimide tends to be lowered, and on the other hand, if the upper limit is exceeded, the colorless transparency of the obtained polyimide tends to be lowered.

In addition, the arylene group that can be selected as R¹⁰ in the general formulas (5) and (6) is preferably at least one of the groups represented by the following general formulas (15) to (19):

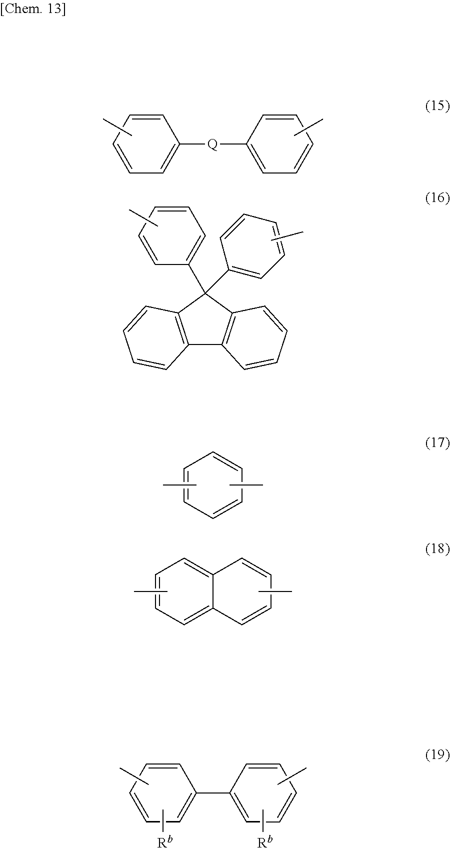

[in the formula (15), Q represents one selected from the group consisting of groups represented by the formulas: —C₆H₄—, —CONH—C₆H₄—NHCO—, —NHCO—C₆H₄—CONH—, —OC₆H₄—CO—C₆H₄—O—, —OCO—C₆H₄—COO—, —OCO—C₆H₄—C₆H₄—COO—, —OCO—, —NC₆H₅—, —CO—C₄H₈N₂—CO—, —C₁₃H₁₀—, —(CH₂)₅, —O—, —S—, —CO—, —CONH—, —SO₂—, —C(CH₃)₂—, —C(CH₃)₂)—, —CH₂—, —(CH₂)₂—, —(CH₂)₃—, —(CH₂)₄, —(CH₂)₅—, —O—C₆H₄—SO₂—C₆H₄—O—, —O—C₆H₄—C(CF₃)₂—C₆H₄—O—, —O—C₆H₄—SO₂—C₆H₄—O—, —C(CH₃)₂—C₆H₄—C(CH₃)₂—, —O—C₆H₄—C₆H₄—O—, and —O—C₆H₄—O—, and R^{b} in the formula (19) represents one selected from the group consisting of a hydrogen atom, a fluorine atom, a methyl group, an ethyl group, and a trifluoromethyl group].

In addition, from the viewpoint that it is possible to obtain a cured product having sufficient levels of heat resistance, transparency, and mechanical strength in a well-balanced manner, the arylene group that can be selected as R¹⁰ in the general formulas (5) and (6) is preferably a divalent group (arylene group) obtained by removing two amino groups from at least one aromatic diamine selected from the group consisting of 4,4′-diaminobenzanilide (abbreviation: DABAN), 4,4′-diaminodiphenyl ether (abbreviation: DDE), 2,2′-bis(trifluoromethyl)benzidine (abbreviation: TFMB), 9,9′-bis(4-aminophenyl)fluorene (abbreviation: FDA), p-diaminobenzene (abbreviation: PPD), 2,2′-dimethyl-4,4′-diaminobiphenyl (also known as m-tolidine), 4,4′-diphenyldiaminomethane (abbreviation: DDM), 4-aminophenyl-4-aminobenzoic acid (abbreviation: BAAB), 4,4′-bis(4-aminobenzamide)-3,3′-dihydroxybiphenyl (abbreviation: BABB), 3,3′-diaminodiphenyl sulfone (abbreviation: 3,3′-DDS), 1,3-bis(4-aminophenoxy)benzene (abbreviation: TPE-R), 4,4′-diaminodiphenyl sulfone (abbreviation: 4,4′-DDS), 3,4′-diaminodiphenyl ether (abbreviation: 3,4-DDE), 2,2-bis(3-amino-4-hydroxyphenyl)-hexafluoropropane (abbreviation: Bis-AP-AF), terephthalic acid bis(4-aminophenyl)ester (abbreviation: BPTP), bis[4-(3-aminophenoxy)phenyl]sulfone (abbreviation: BAPS-M), 1,3-bis(3-aminophenoxy)benzene (abbreviation: APB-N), 2,2-bis(3-amino-4-hydroxyphenyl)propane (abbreviation: BAPA), and 2,2-bis(3-amino-4-hydroxyphenyl)sulfone (abbreviation: BPS-DA), and is more preferably a divalent group (arylene group) obtained by removing two amino groups from at least one aromatic diamine selected from the group consisting of DABAN, DDE, TFMB, FDA, PPD, m-tolidine, DDM, BAAB, BABB, 3,3′-DDS, TPE-R, and 4,4′-DDS.

Y in the general formulas (5) and (6) each independently represent one selected from the group consisting of a hydrogen atom, alkyl groups having 1 to carbon atoms (preferably 1 to 3 carbon atoms), and alkylsilyl groups having 3 to 9 carbon atoms. Such Y can be changed by appropriately changing the type of the substituent and the introduction rate of the substituent by appropriately changing the production conditions thereof. When such Y is each a hydrogen atom (when it is a repeating unit of so-called polyamic acid), the production of polyimide tends to be easier.

In addition, when Y in the general formulas (5) and (6) is an alkyl group having 1 to 6 carbon atoms (preferably to 3 carbon atoms), the storage stability of the polyimide precursor resin tends to be better. In addition, when Y is an alkyl group having 1 to 6 carbon atoms (preferably 1 to 3 carbon atoms), Y is more preferably a methyl group or an ethyl group. In addition, when Y in the general formulas (5) and (6) is an alkylsilyl group having 3 to 9 carbon atoms, the solubility of the polyimide precursor resin tends to be better. When Y is an alkylsilyl group having 3 to 9 carbon atoms as described above, Y is more preferably a trimethylsilyl group or a t-butyldimethylsilyl group.

Regarding Y of each formula in the repeating unit (I), the introduction rate of a group other than a hydrogen atom (alkyl group and/or alkylsilyl group) is not particularly limited, but when at least part of Y in the formula is an alkyl group and/or an alkylsilyl group, preferably, 25% or more (more preferably 50% or more and further preferably 75% or more) of the total amount of Y in the repeating unit (I) is an alkyl group and/or an alkylsilyl group (note that, in this case, Y other than the alkyl group and/or the alkylsilyl group is a hydrogen atom). For each of Y in the repeating unit (I), if 25% or more of the total amount is an alkyl group and/or an alkylsilyl group, the storage stability of the polyimide precursor resin tends to be better.

In addition, in the general formulas (5) and (6), one of the bonder represented by *1 and the bonder represented by *2 is bonded to the carbon atom a (carbon atom with the symbol a) forming the norbornane ring, and the other of the bonder represented by *1 and the bonder represented by *2 is bonded to the carbon atom b (carbon atom with the symbol b) forming the norbornane ring. In addition, in the general formulas (5) and (6), one of the bonder represented by *3 and the bonder represented by *4 is bonded to the carbon atom c (carbon atom with the symbol c) forming the norbornane ring, and the other of the bonder represented by *3 and the bonder represented by *4 is bonded to the carbon atom d (carbon atom with the symbol d) forming the norbornane ring. Then, in the general formula (6), each of the bonders represented by *1 to *4 has an exo conformation with respect to the norbornane ring to which the bonder is bonded. As described above, in the general formula (6), each of the bonders represented by *1 to *4 has a structure taking an exo conformation with respect to the bonded norbornane ring, and in the present invention, the repeating unit having such a structure represented by the general formula (6) is treated as a repeating unit having an “exo/exo type three-dimensional structure” among the repeating units represented by the general formula (5) (repeating units capable of taking various three-dimensional structures).

The polyimide precursor resin of the present invention is a polyimide precursor resin comprising the repeating unit (I) represented by the above general formula (5), wherein 60% by mass or more of the repeating unit (I) contained in the polyimide precursor resin is a repeating unit having an exo/exo type three-dimensional structure represented by the above general formula (6). Here, in addition to the repeating unit of exo/exo type three-dimensional structure, the repeating unit (I) represented by the general formula (5) may include a repeating unit of endo/endo type three-dimensional structure. Note that, regarding the three-dimensional structure of such a repeating unit (I), the “endo/endo type” means, to explain based on the above general formula (5), a three-dimensional structure in the case where each of the bonders represented by *1 to *4 has an endo conformation with respect to the norbornane ring to be bonded (unlike the above general formula (6), the bonders represented by *1 to *4 are bonded at endo positions) (note that the repeating unit of such an endo/endo type three-dimensional structure can be easily prepared by using the endo/endo type tetracarboxylic dianhydride represented by the above general formula (2′) as a monomer).

As described above, the repeating unit (I) may include multiple kinds of repeating units having different three-dimensional structures, and the polyimide precursor resin of the present invention contains the repeating unit (I) represented by the above general formula (5), wherein the content of the repeating unit having an exo/exo type three-dimensional structure (repeating unit represented by the general formula (6)) in the repeating unit (I) is 60% by mass or more. If the content of the repeating unit having such an exo/exo type three-dimensional structure is less than the above lower limit, the linear expansion coefficient of the obtained polyimide cannot be set to a lower value when induced into the polyimide. In addition, from the viewpoint that the linear expansion coefficient of the obtained polyimide can be set to a further lower value when induced into the polyimide, the content of the repeating unit having such an exo/exo type three-dimensional structure in the repeating unit (I) is more preferably 70% by mass or more (further preferably 80% by mass or more, and particularly preferably 90% by mass or more).

Note that, when the repeating unit (I) includes a repeating unit having a different three-dimensional structure other than the repeating unit having an exo/exo type three-dimensional structure, the repeating unit having such a different three-dimensional structure is preferably a repeating unit having an endo/endo type three-dimensional structure.

In addition, in such a polyimide precursor resin, the content of the repeating unit (I) represented by the above general formula (5) is more preferably 50 to 100 mol % (more preferably 70 to 100 mol %, and further preferably 80 to 100 mol %). In addition, such a polyimide precursor resin may contain a different repeating unit as long as the effects of the present invention are not impaired. Examples of such a different repeating unit include repeating units derived from tetracarboxylic dianhydrides other than the tetracarboxylic dianhydride represented by the above general formula (1). As the tetracarboxylic dianhydrides other than the tetracarboxylic dianhydride represented by the general formula (1), known tetracarboxylic dianhydrides can be appropriately used, and for example, one described in paragraph [0230] of International Publication No. WO2015/163314 may be appropriately used.

The polyamic acid has an intrinsic viscosity [η] of preferably 0.05 to 3.0 dL/g, and more preferably 0.1 to 2.0 dL/g. If the intrinsic viscosity [η] is lower than 0.05 dL/g, the obtained film tends to be brittle, when a polyimide in the form of a film is produced by using this polyamic acid. Meanwhile, if the intrinsic viscosity [η] exceeds 3.0 dL/g, the viscosity is so high that the processability decreases, for example, making it difficult to form a uniform film when a film is produced. In addition, the intrinsic viscosity [η] employed is a value obtained by preparing a measurement sample (solution) in which the polyamic acid is dissolved in N,N-dimethylacetamide to have a concentration of 0.5 g/dL, and measuring the viscosity of the measurement sample using a kinematic viscometer under a temperature condition of 30° C. Note that, as the kinematic viscometer, an automatic viscometer manufactured by RIGO CO., LTD. (trade name: “VMC-252”) can be used.

In addition, as a method for producing such a polyimide precursor resin of the present invention, a method for producing a polyimide precursor resin by reacting the tetracarboxylic anhydride of the present invention with an aromatic diamine represented by the formula: H₂N—R¹⁰—NH₂ [R¹⁰ in the formula has the same definition as R¹⁰ in the general formulas (5) and (6) ] can be mentioned as a preferable method. As such an aromatic diamine, a known one (for example, the aromatic diamine described in paragraph [0039] of Japanese Unexamined Patent Application Publication No. 2018-44180, or the like) can be appropriately used. In addition, the conditions for reacting the tetracarboxylic anhydride with the aromatic diamine are not particularly limited, and known conditions such as those used when preparing the polyamic acid can be appropriately employed (for example, the conditions (such as solvent and reaction temperature) used in the methods described in paragraphs [0215] to of International Publication No. WO2015/163314 can be appropriately employed). Note that, when the tetracarboxylic anhydride of the present invention is reacted with the aromatic diamine, the repeating unit (I) can be a repeating unit of a polyamic acid in which Y is each a hydrogen atom. Here, as a production method in the case of producing a polyimide precursor resin containing such a repeating unit (I) that Y is other than a hydrogen atom, for example, it is possible to appropriately employ the same production method as the method described in paragraphs [0165] to [0174] of International Publication No. WO2018/066522 except that the above tetracarboxylic anhydride of the present invention is used as the tetracarboxylic dianhydride. In addition, when the tetracarboxylic anhydride of the present invention is reacted with the aromatic diamine to form a polyimide precursor resin, a repeating unit having an exo/exo type three-dimensional structure can be contained at the same ratio as the content ratio of the exo/exo type tetracarboxylic anhydride contained in the tetracarboxylic anhydride of the present invention (the three-dimensional structure is basically maintained during the reaction).

Note that the polyimide precursor resin (preferably polyamic acid) of the present invention may be contained in an organic solvent and used as a polyimide precursor resin solution (varnish). The content of the polyimide precursor resin in such a polyimide precursor resin solution is not particularly limited, but is preferably 1 to 80% by mass, and more preferably 5 to 50% by mass. If the content is less than the above lower limit, it tends to be difficult to use it as a varnish for producing a polyimide film. Meanwhile, if the upper limit is exceeded, it tends to be difficult to use it as a varnish for producing a polyimide film. Note that such a polyimide precursor resin solution can be suitably used as a resin solution (varnish) for producing the polyimide of the present invention, and can be suitably used for producing polyimides having various shapes. For example, a film-shaped polyimide can be easily produced by applying such a polyimide precursor resin solution on various substrates, followed by imidization and curing. Note that the organic solvent used for such a polyimide precursor resin solution (varnish) is not particularly limited, and known ones can be appropriately used. For example, the solvents and the like described in paragraph and paragraphs [0133] to [0134] of International Publication No. WO2018/066522 can be appropriately used.

### [Polyimide]

The polyimide of the present invention is a polyimide comprising a repeating unit (A) represented by the above general formula (7), wherein 60% by mass or more of the repeating unit (A) contained in the polyimide is a repeating unit having an exo/exo type three-dimensional structure represented by the above general formula (8).

Such A and R^{a}s in the general formula (7) and general formula (8) have the same definition as A and R^{a}s in the general formulas (1) and (2), respectively (the suitable ones and suitable conditions (conditions for conformation of A and the like) are also the same), and R¹⁰ in the above general formula (7) and general formula (8) has the same definition as R¹⁰ in the above general formulas (5) and (6) (the suitable ones and suitable conditions are also the same).

The polyimide of the present invention is a polyimide precursor resin containing a repeating unit (A) represented by the above general formula (7), wherein 60% by mass or more of the repeating unit (A) contained in the polyimide precursor resin is a repeating unit having an exo/exo type three-dimensional structure represented by the above general formula (8). Here, in addition to the repeating unit of exo/exo type three-dimensional structure, the repeating unit (A) represented by the general formula (7) may include a repeating unit of endo/endo type three-dimensional structure. Note that, regarding the three-dimensional structure of such a repeating unit (A), the “endo/endo type” means that any imide ring bonded to the norbornane ring in the repeating unit represented by the general formula (7) has an endo conformation with respect to the norbornane ring to be bonded (note that the repeating unit of such an endo/endo type three-dimensional structure can be easily prepared by using the endo/endo type tetracarboxylic dianhydride represented by the above general formula (2′) as a monomer and reacting it with an aromatic diamine).

As described above, the repeating unit (A) may include multiple kinds of repeating units having different three-dimensional structures, and the polyimide of the present invention contains the repeating unit (A) represented by the above general formula (7), wherein the content of the repeating unit having an exo/exo type three-dimensional structure (repeating unit represented by the general formula (8)) in the repeating unit (A) is 60% by mass or more. If the content of the repeating unit having such an exo/exo type three-dimensional structure is less than the above lower limit, the linear expansion coefficient of the polyimide cannot be set to a lower value. In addition, from the viewpoint that the linear expansion coefficient of the polyimide can be set to a further lower value, the content of the repeating unit having such an exo/exo type three-dimensional structure in the repeating unit (A) is more preferably 70% by mass or more (further preferably 80% by mass or more, and particularly preferably 90% by mass or more).

Note that, when the repeating unit (A) includes a repeating unit having a different three-dimensional structure other than the repeating unit having an exo/exo type three-dimensional structure, the repeating unit having such a different three-dimensional structure is preferably a repeating unit having an endo/endo type three-dimensional structure.

In addition, in such a polyimide, the content of the repeating unit (A) represented by the above general formula (7) is more preferably 50 to 100 mol % (more preferably 70 to 100 mol %, and further preferably 80 to 100 mol %). In addition, such a polyimide may contain a different repeating unit as long as the effects of the present invention are not impaired. Examples of such a different repeating unit include repeating units derived from tetracarboxylic dianhydrides other than the tetracarboxylic dianhydride represented by the above general formula (1). As the tetracarboxylic dianhydrides other than the tetracarboxylic dianhydride represented by the general formula (1), known tetracarboxylic dianhydrides can be appropriately used, and for example, one described in paragraph [0230] of International Publication No. WO2015/163314 may be appropriately used.

In addition, such a polyimide has a glass transition temperature (Tg) of preferably 250° C. or higher, further preferably 270° C. or higher, and particularly preferably 320 to 500° C. If the glass transition temperature (Tg) is less than the lower limit, it tends to be difficult to obtain sufficiently high heat resistance. On the other hand, if the upper limit is exceeded, it tends to be difficult to produce a polyimide having such characteristics. Note that such a glass transition temperature (Tg) can be measured using a thermomechanical analyzer (under the trade name of “TMA 8311” manufactured by Rigaku Corporation).

In addition, such a polyimide has a 5% weight loss temperature of preferably 350° C. or higher, and more preferably 450 to 600° C. Note that such 5% weight loss temperature can be determined by gradually heating from room temperature (25° C.) while flowing nitrogen gas in a nitrogen gas atmosphere, and measuring the temperature at which the weight of the sample used is reduced by 5%. Moreover, such a polyimide has a softening temperature (Tg) of preferably 250° C. or higher, further preferably 270° C. or higher, and particularly preferably 320 to 500° C. Note that such a softening temperature can be measured in a penetration mode using a thermomechanical analyzer (under the trade name of “TMA 8311” manufactured by Rigaku Corporation). In addition, such a polyimide has a thermal decomposition temperature (Td) of preferably 400° C. or higher, and more preferably 450 to 600° C. Note that the thermal decomposition temperature (Td) can be determined by measuring the temperature at an intersection of tangent lines drawn to decomposition curves before and after thermal decomposition using a TG/DTA220 thermogravimetric analyzer (manufactured by SII NanoTechnology Inc.) under a nitrogen atmosphere under a condition of a rate of temperature rise of 10° C./minute.

Moreover, the polyimide preferably has a number average molecular weight (Mn) of 1000 to 1000000 in terms of polystyrene. In addition, the polyimide preferably has a weight average molecular weight (Mw) of 1000 to 5000000 in terms of polystyrene. Moreover, the polyimide preferably has a molecular weight distribution (Mw/Mn) of 1.1 to 5.0. Note that the molecular weights (Mw and Mn) of the polyimide and the distribution (Mw/Mn) of the molecular weights can be determined by using a gel permeation chromatograph as a measuring apparatus and converting the measured data to that of polystyrene.

In addition, the polyimide is preferably one having a sufficiently high transparency when formed into a film, and the film has a total luminous transmittance of more preferably 80% or higher (further preferably 85% or higher, and particularly preferably 87% or higher). Such a total luminous transmittance can be obtained by performing a measurement in accordance with JIS K7361-1 (issued in 1997).

In addition, the polyimide has a linear expansion coefficient of preferably 0 to 70 ppm/K, more preferably 0 to 60 ppm/K, and further preferably 5 to 40 ppm/K. If the linear expansion coefficient exceeds the upper limit, the polyimide tends to be easily peeled off because of thermal history when a composite material is formed by combining the polyimide with a metal or an inorganic material having a linear expansion coefficient in a range from 5 to 20 ppm/K. Meanwhile, if it is less than the lower limit, the polyimide is too rigid, the elongation at break is low, and the flexibility tends to decrease. The linear expansion coefficient of the polyimide is as follows. Specifically, a measurement sample is prepared by forming a polyimide film in a size of 20 mm in length and 5 mm in width (the thickness of the film is not particularly limited because it does not affect the measured value, but it is preferably 5 to 80 μm). Then, the change in length of the sample in the longitudinal direction is measured from 50° C. to 200° C. by using a thermomechanical analyzer (manufactured by Rigaku Corporation under the trade name of “TMA 8311,” for example) as a measuring apparatus and by employing a condition of a rate of temperature rise of 5° C./minute under a nitrogen atmosphere in a tensile mode (49 mN). The average value of changes in length per Celsius degree is determined for the temperature range from 100° C. to 200° C. The thus obtained value is employed as the linear expansion coefficient.

In addition, the polyimide has a haze (turbidity) of 5 to 0 (further preferably 4 to 0, and particularly preferably 3 to 0). Moreover, the polyimide has a yellowness index (YI) of 5 to 0 (further preferably 4 to 0, and particularly preferably 3 to 0). The haze (turbidity) can be determined by measuring in accordance with JIS K7136 (issued in 2000), and the yellowness index (YI) can be determined by measuring in accordance with ASTM E313-05 (issued in 2005).

In addition, a method for producing such a polyimide of the present invention is not particularly limited, and for example, a method for producing a polyimide by reacting the tetracarboxylic anhydride of the present invention with an aromatic diamine represented by the formula: H₂N—R¹⁰—NH₂ [R¹⁰ in the formula has the same definition as R¹⁰ in the general formulas (5) and (6) ] can be mentioned as a preferable method. As the conditions for reacting the tetracarboxylic anhydride of the present invention with the aromatic diamine, it is possible to appropriately employ the conditions employed in the known method for producing polyimide by reacting a tetracarboxylic anhydride with a diamine. As described above, it is possible to produce the polyimide of the present invention in the same manner as the known method for producing a polyimide by reacting a tetracarboxylic anhydride with a diamine except for using the tetracarboxylic anhydride of the present invention and the aromatic diamine as the monomers. Note that, in the case of employing the method for producing polyimide by reacting the tetracarboxylic anhydride of the present invention with the aromatic diamine, a polyimide may be produced by reacting the tetracarboxylic anhydride of the present invention with the aromatic diamine to prepare the polyamic acid of the present invention, followed by imidization thereof. The imidization method is not particularly limited, and conditions and the like employed in a known method capable of imidizing a polyamic acid (for example, the method as described in paragraphs [0238] to [0262] of International Publication No. WO2015/163314) can be appropriately employed. Note that, when the tetracarboxylic anhydride of the present invention is reacted with the aromatic diamine to form a polyimide, a repeating unit having an exo/exo type three-dimensional structure can be contained at the same ratio as the content ratio of the exo/exo type tetracarboxylic anhydride contained in the tetracarboxylic anhydride of the present invention (the three-dimensional structure is basically maintained during the reaction).

Note that the polyimide of the present invention has sufficiently high transparency, a sufficiently low linear expansion coefficient, and a sufficiently high level of heat resistance. Therefore, for example, it can be appropriately used for applications such as flexible wiring board films, liquid crystal orientation films, transparent conductive films for organic EL, film for organic EL lighting, flexible substrate films, flexible organic EL substrate films, flexible transparent conductive films, transparent conductive films, transparent conductive films for organic thin-film solar cells, transparent conductive films for dye-sensitized type solar cells, flexible gas barrier films, touch panel films, flexible display front films, flexible display back films, polyimide belts, coating agents, barrier films, sealants, interlayer insulation materials, passivation films, TAB tapes, FPCs, COFs, optical fibers, color filter base materials, semiconductor coating agents, heat-resistant insulating tapes, and enameled wires.

### EXAMPLES

Hereinafter, the present invention is described in more detail based on Examples and Comparative Examples, but the present invention is not limited to the following Examples.

### Synthesis Example 1

To a 1 L reaction vessel, cis-5s-norbornene-exo-2,3-dicarboxylic anhydride (100 g, 0.609 mol, exo form:endo form=98:2) manufactured by Mancherster Organics, methanol (500 mL), and concentrated hydrochloric acid (5.0 mL) having a concentration of 37% by mass were sequentially added under an argon stream to obtain a mixture liquid. Then, the mixture liquid was stirred under reflux conditions (internal temperature: 65° C.) for 4 hours to obtain a reaction liquid. After the reaction under reflux conditions for 4 hours in this way (after completion of the reaction), GC measurement was performed on the reaction liquid to confirm the disappearance of the raw material cis-5s-norbornene-exo-2,3-dicarboxylic anhydride.

Then, methanol was distilled off from the reaction liquid under reduced pressure using a rotary evaporator to obtain a liquid product. Next, the liquid product was dissolved in ethyl acetate (500 mL) and transferred to a separatory funnel. The liquid product was washed twice with saturated sodium hydrogen carbonate aqueous solution (200 mL) and then once with water (200 mL) to obtain an organic layer. Then, ethyl acetate was distilled off from the organic layer under reduced pressure using a rotary evaporator, thereby obtaining cis-5-norbornene-exo-2,3-dimethyl dicarboxylate (120 g, yield: 94%, exo form:endo form=100:0). The structure of the product was identified by ¹H-NMR and ¹³C-NMR. Note that, regarding the above product, the “exo form” means that all the groups represented by the formula: —COOMe have an exo conformation with respect to the norbornene ring to be bonded, and on the other hand, the “endo form” means that all the groups represented by the formula: —COOMe have an endo conformation with respect to the norbornene ring to be bonded. The reaction formula of the reaction used in the production of such a product is presented below.

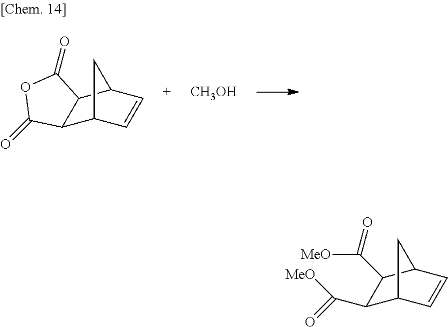

### Example 1

Into a 3 L reaction vessel, palladium acetate (118 mg, 0.524 mmol), tri-o-tolylphosphine (159 mg, 0.524 mmol), and N,N-dimethylformamide (596 mL) were sequentially charged under an argon stream, followed by stirring at an internal temperature of 50 to 56° C. for 30 minutes. Next, to the inside of the reaction vessel, cis-5-norbornene-exo-2,3-dimethyl dicarboxylate (110 g, 0.523 mol, ratio of exo form: 100 mol %) obtained in Synthesis Example 1, 1, 4-dibromobenzene (143 g, 0.262 mol), triethylamine (106 g, 1.05 mol), formic acid (48.3 g, 0.262 mol), and N,N-dimethylformamide (660 mL) were further added to obtain a mixture liquid. Then, the mixture liquid was heated to an internal temperature of 80° C. and stirred for 8 hours to obtain a reaction liquid. After the reaction while stirring for 8 hours in this way (after the completion of the reaction), the reaction liquid was allowed to cool until the temperature thereof reached room temperature.

Next, the reaction liquid was moved to a separatory funnel, toluene (2.62 L) and water (1.05 L) were added, and liquid separation washing was performed. Next, the organic layer thus obtained was washed twice with hydrochloric acid (520 mL) having a concentration of 5% by mass, twice with a saturated sodium hydrogen carbonate aqueous solution (520 mL), and further washed twice with water (520 mL). Then, the black insoluble matter in the intermediate layer was removed by Celite filtration. The obtained filtrate was heated under the condition of a water bath temperature of 60° C. and concentrated to obtain a crude product.

Next, the crude product (135.4 g) thus obtained was added with ethyl acetate (108 mL) to obtain a mixture liquid, and then, the mixture liquid was added with cyclohexane (1.05 L) while heating and stirring under the condition of a water bath temperature of 60° C. to prepare a solution, and crystallization was carried out as follows. Specifically, the solution was prepared as described above, which was heated and stirred under the condition of a water bath temperature of 50° C., and the crystals were precipitated as a precipitation product by gradual cooling to room temperature while continuing the stirring (crystallization). After filtering the precipitation product obtained by such a crystallization step, the obtained filtrate was washed with cyclohexane (211 mL) and then dried under reduced pressure at 80° C. for 5 hours to obtain a white product. In order to analyze the absolute structure of the product thus obtained, one-dimensional NMR (¹H and ¹³C) and two-dimensional NMR (DEPT 135, DQF COSY, HMQC, HMBC, NOESY) measurements were preformed, and the product was found to be an ester compound having a structure represented by the following formula (yield 49%):

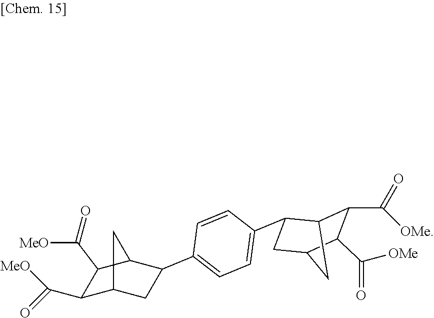 [SMILES: COC(=O)C1C2CC(c3ccc(C4CC5CC4C(C(=O)OC)C5C(=O)OC)cc3)C(C2)C1C(=O)OC]

As described above, analysis of the absolute structure revealed that the product obtained was an exo/exo type ester compound (tetramethyl exo,exo-5,5′-(1,4-phenylene)bis(bicyclo[2.2.1]heptane-2,3-exo-dicarboxylate) in which each methyl ester group had a structure taking an exo conformation with respect to the norbornane ring to be bonded. Note that it was also found that, in the exo/exo type ester compound, the benzene ring had an exo conformation with respect to both norbornane rings.

### Example 2

Into a 300 mL reaction vessel, the exo/exo type ester compound (13.0 g, 26.1 mmol) obtained in Example 1, acetic acid (185 g), and acetic acid solution of 10 mass % trifluoromethanesulfonic acid prepared in advance (1.96 g, trifluoromethanesulfonic acid: 1.30 mmol) were sequentially charged under an argon stream to obtain a reaction solution. Then, while heating and refluxing the reaction solution, an operation of adding 18 g of acetic acid while extracting 18 g of the distillate was carried out every hour using a Dean-Stark tube. Such an operation continued until 6 hours had passed since the extraction of 18 g of the distillate was started. After operating for 6 hours in this manner, the heating and refluxing were stopped, and the reaction solution was allowed to cool to room temperature, and allowed to stand overnight because no precipitation product was precipitated. The next day, when the reaction solution after allowed to stand overnight was confirmed again, a white precipitation product had been precipitated in the reaction solution, so that it was filtered and washed once with acetic acid (20 mL) and once with ethyl acetate (20 mL) to obtain a filtrate. Next, the filtrate was dried under reduced pressure at 80° C. for 5 hours to obtain a white product. In order to analyze the absolute structure of the white product thus obtained, one-dimensional NMR (¹H and ¹³C) and two-dimensional NMR (DEPT 135, DQF COSY, HMQC, HMBC, NOESY) measurements were preformed, and the product was found to be an acid dianhydride having a structure represented by the following formula (yield 58%):

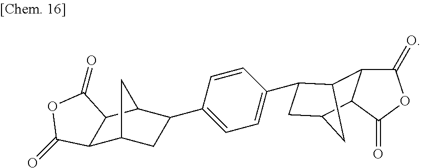 [SMILES: O=C1OC(=O)C2C3CC(CC3c3ccc(C4CC5CC4C4C(=O)OC(=O)C54)cc3)C12]

As described above, analysis of the absolute structure revealed that the product was an exo/exo type tetracarboxylic dianhydride (exo,exo-5,5′-(1,4-phenylene)bis(bicyclo[2.2.1]heptane-2,3-exo-dicarboxylic anhydride) in which each acid anhydride group had a structure taking an exo conformation with respect to the norbornane ring to be bonded. Note that it was also found that, in the exo/exo type tetracarboxylic dianhydride, the benzene ring had an exo conformation with respect to both norbornane rings. Moreover, when liquid chromatography (LC) analysis was performed, the LC purity of the product (tetracarboxylic dianhydride) was 96 area %.

Next, the exo/exo type tetracarboxylic dianhydride (16.9 g) thus obtained was charged into a glass tube oven, and then the pressure was reduced, and heating was started after the degree of vacuum reached 6.5×10⁻⁴ Pa. By such heating, the acid dianhydride was first melted when the temperature reached 250° C., and then, evaporation started when the temperature reached 270° C., and the degree of vacuum increased to 4.3×10⁻³ Pa. Then, a distillation operation was carried out to obtain 15.3 g of a purified product (yield: 98%). Note that it was confirmed by ¹H-NMR measurement and LC analysis that there were no impurities (LC purity: >99 area %). In this way, a purified exo/exo type tetracarboxylic dianhydride was obtained. Hereinafter, the tetracarboxylic dianhydride thus obtained is sometimes referred to as “exo/exo type BzDA.”

### Synthesis Example 2

To a 1 L reaction vessel, 5-norbornene-2,3-dicarboxylic anhydride (1,150 g, 7.01 mol, exo form:endo form=0:100) manufactured by Wako Pure Chemical Industries, Ltd., methanol (5.75 mL), and concentrated hydrochloric acid (57.5 mL) having a concentration of 37% by mass were sequentially added under an argon stream to obtain a mixture liquid. Then, the mixture liquid was stirred under reflux conditions (internal temperature: 65° C.) for 4 hours to obtain a reaction liquid. After the reaction under reflux conditions for 4 hours in this way (after completion of the reaction), GC measurement was performed on the reaction liquid to confirm the disappearance of the raw material 5-norbornene-2,3-dicarboxylic anhydride.

Then, methanol was distilled off from the reaction liquid under reduced pressure using a rotary evaporator to obtain a liquid product. Next, the liquid product was dissolved in ethyl acetate (5.8 L) and transferred to a separatory funnel. The liquid product was washed twice with saturated sodium hydrogen carbonate aqueous solution (2.3 L) and then once with water (2.3 L) to obtain an organic layer. Then, ethyl acetate was distilled off from the organic layer under reduced pressure using a rotary evaporator, thereby obtaining cis-5-norbornene-endo-2,3-dimethyl dicarboxylate (1,404 g, yield: 95%, exo form: endo form=0:100). Regarding the above product, the “exo form” means that all the groups represented by the formula: —COOMe have an exo conformation with respect to the norbornene ring to be bonded, and on the other hand, the “endo form” means that all the groups represented by the formula: —COOMe have an endo conformation with respect to the norbornene ring to be bonded. Note that the structure of the product was also identified by ¹H-NMR.

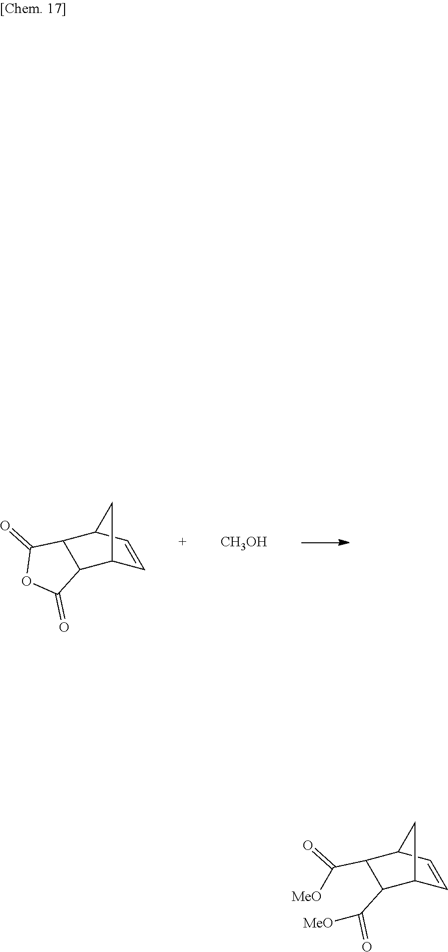 [SMILES: O=C1OC(=O)C2C3C=CC(C3)C12 ; "CH3OH" ; COC(=O)C1C2C=CC(C2)C1C(=O)OC]

### Comparative Example 1

Into a 3 L reaction vessel, palladium acetate (1.20 g, 5.35 mmol), tri-o-tolylphosphine (1.63 g, 5.35 mmol), and N,N-dimethylformamide (4.28 L) were sequentially charged under an argon stream, followed by stirring at an internal temperature of 50 to 56° C. for 30 minutes. Next, to the inside of the reaction vessel, cis-5-norbornene-endo-2,3-dimethyl dicarboxylate (1,125 g, 5.35 mol) obtained in Synthesis Example 2, 1,4-dibromobenzene (757 g, 3.21 mol), triethylamine (1,083 g, 10.7 mol), formic acid (493 g, 10.7 mol), and N,N-dimethylformamide (4.28 L) were further added to obtain a mixture liquid. Then, the mixture liquid was heated to an internal temperature of 80° C. and stirred for 8 hours to obtain a reaction liquid. After the reaction while stirring for 8 hours in this way (after the completion of the reaction), the reaction liquid was allowed to cool until the temperature thereof reached room temperature.

Next, the reaction liquid was moved to a separatory funnel, toluene (26.9 L) and water (10.7 L) were added, and liquid separation washing was performed. The organic layer obtained was washed twice with hydrochloric acid (5.3 L) having a concentration of 5% by mass, twice with a saturated sodium hydrogen carbonate aqueous solution (5.3 L), and further washed twice with water (5.3 L). Then, the black insoluble matter in the intermediate layer was removed by Celite filtration. The obtained filtrate was heated under the condition of a water bath temperature of 60° C., and the reaction solution was concentrated under reduced pressure to 2,000 g to obtain a concentrated liquid. Then, toluene was added to the concentrated liquid and diluted to obtain a solution. The total amount of the solution thus obtained was 2,940 g.

Next, the solution was divided into two (1,470 g×2), and when cyclohexane (14.8 L) was added to each solution while heating each solution under the condition of a water bath temperature of 60° C., a white precipitation product was formed in each solution. Each of the above solutions with a precipitation product thus produced therein was then stirred for 30 minutes while heating under the condition of a water bath temperature of 50° C., and then allowed to cool to room temperature. Next, the precipitation product was filtered from each of the resulting solutions, the resulting filtrate was washed with cyclohexane (1.07 L) and then dried under reduced pressure at 80° C. for 5 hours to obtain a white product. In order to analyze the absolute structure of the product obtained, one-dimensional NMR (¹H and ¹³C) and two-dimensional NMR (DEPT 135, DQF COSY, HMQC, HMBC, NOESY) measurements were preformed, and the product was found to be an ester compound having a structure represented by the following formula (yield 51%):

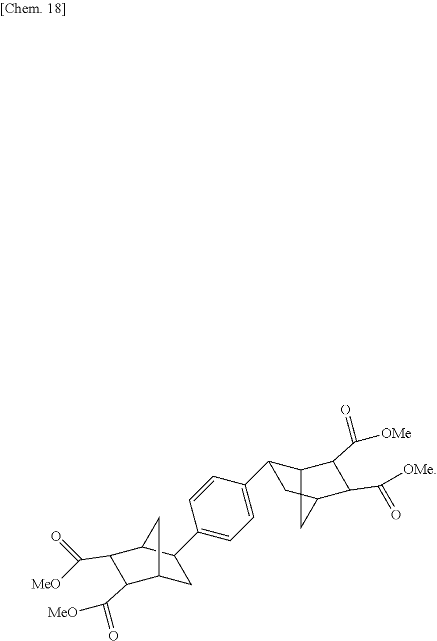 [SMILES: COC(=O)C1C2CC(c3ccc(C4CC5CC4C(C(=O)OC)C5C(=O)OC)cc3)C(C2)C1C(=O)OC]

As described above, analysis of the absolute structure revealed that the product was an endo/endo type ester compound (tetramethyl exo,exo-5,5′-(1,4-phenylene)bis(bicyclo[2.2.1]heptane-2,3-endo-dicarboxylate) in which each methyl ester group had a structure taking an endo conformation with respect to the norbornane ring to be bonded. Note that it was also found that, in the endo/endo type ester compound, the benzene ring had an exo conformation with respect to both norbornane rings.

### Comparative Example 2

Into a 20 L reaction vessel, the endo/endo type ester compound (650 g, 1.30 mol) obtained in Comparative Example 1, acetic acid (9.34 kg), and acetic acid solution of 10 mass % trifluoromethanesulfonic acid prepared in advance (9.78 g, trifluoromethanesulfonic acid: 65.2 mmol) were sequentially charged under an argon stream to obtain a reaction solution. Then, while heating and refluxing the reaction solution, an operation of adding 1100 g of acetic acid while extracting 1100 g of the distillate was carried out every hour using a Dean-Stark tube. Such an operation continued until 6 hours had passed since the extraction of the distillate was started. Note that 1 hour after the start of heating and refluxing, a white precipitate product was formed in the reaction solution. In this way, after continuing the above operation for 6 hours, the heating and refluxing were stopped, and the reaction solution was allowed to cool to room temperature, and allowed to stand overnight. The next day, the white precipitate was filtered from the reaction solution that had been allowed to stand overnight, and was washed once with acetic acid (1.9 L) and five times with ethyl acetate (1.9 L) to obtain a filtrate. Next, the filtrate was dried under reduced pressure at 80° C. for 5 hours to obtain a white product. In order to analyze the absolute structure of the product thus obtained, one-dimensional NMR (¹H and ¹³C) and two-dimensional NMR (DEPT 135, DQF COSY, HMQC, HMBC, NOESY) measurements were preformed, and the product was found to be an acid dianhydride having a structure represented by the following formula (yield 86%):

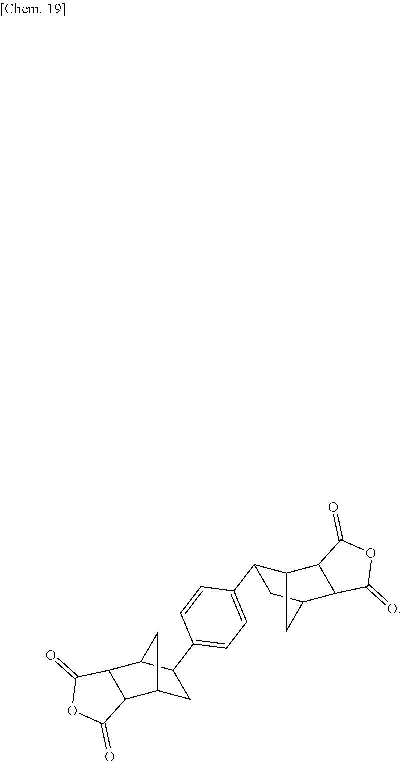 [SMILES: O=C1OC(=O)C2C3CC(CC3c3ccc(C4CC5CC4C4C(=O)OC(=O)C54)cc3)C12]

As described above, analysis of the absolute structure revealed that the product was an endo/endo type tetracarboxylic dianhydride in which each acid anhydride group had a structure taking an endo conformation with respect to the norbornane ring to be bonded. Note that it was also found that, in the endo/endo type tetracarboxylic dianhydride, the benzene ring had an exo conformation with respect to both norbornane rings. In addition, when liquid chromatography (LC) analysis was performed, the LC purity of the product was 99%. The endo/endo type tetracarboxylic dianhydride thus obtained is hereinafter referred to as “endo/endo type BzDA” in some cases.

### [On Solubility of Tetracarboxylic Dianhydride in Organic Solvent]

As the samples, the tetracarboxylic dianhydride (exo/exo type BzDA) obtained in Example 2 and the tetracarboxylic dianhydride (endo/endo type BzDA) obtained in Comparative Example 2 were separately used to confirm the solubility of each tetracarboxylic dianhydride in an organic solvent as follows. Specifically, after 50 mg of the sample was added to a screw tube, an organic solvent was added little by little into the screw tube, and the amount of the sample dissolved was visually confirmed. Note that, as the organic solvents, N,N′-dimethylacetamide and N-methyl-2-pyrrolidone were used to confirm the solubility in the solvents. As a result of the test, the exo/exo type BzDA obtained in Example 2 was easily dissolved in each solvent (N,N′-dimethylacetamide, N-methyl-2-pyrrolidone), and it was found that the use of these solvents (N,N′-dimethylacetamide, N-methyl-2-pyrrolidone) made it possible to sufficiently prepare a solution having a concentration of 5% by mass or more. On the other hand, it was found that the endo/endo type BzDA obtained in Comparative Example 2 had low solubility in each solvent (N,N′-dimethylacetamide, N-methyl-2-pyrrolidone), the use of N,N′-dimethylacetamide did not make it possible to prepare a solution having a concentration of 1% by mass or more, and even the use of N-methyl-2-pyrrolidone did not make it possible to prepare a solution having a concentration of 3.5% by mass or more. From these results, it was found that the tetracarboxylic dianhydride having an exo/exo type three-dimensional structure (exo/exo type BzDA: Example 2) has extremely high solubility in organic solvents.

### Example 3

Under a nitrogen atmosphere, into a 15 mL screw tube, 0.560 g (2.46 mmol) of 4,4′-diaminobenzanilide (DABAN) was introduced as an aromatic diamine, and also, 1.01 g (2.46 mmol) of the exo/exo type BzDA obtained in Example 2 was introduced as a tetracarboxylic dianhydride. Next, 6.2 g of tetramethylurea (TMU) as a solvent was added into the screw tube to obtain a mixture liquid. Next, the obtained mixture liquid was stirred under a nitrogen atmosphere and under a temperature condition of room temperature for 5 days to obtain a reaction liquid (varnish) (the step of obtaining such a reaction liquid (varnish) is hereinafter referred to as the “varnish preparation step”). Note that it is found that the varnish contains a polyamic acid in which a repeating unit (I) is contained represented by the general formula (5) derived from the exo/exo type BzDA used, and in which, in the repeating unit (I), the content of the repeating unit having an exo/exo type three-dimensional structure represented by the above general formula (6) is 100% by mass (note that, in the formulas (5) and (6), A is a p-phenylene group, R¹⁰ is a divalent group obtained by removing two amino groups from DABAN, and R^{a} and Y are both hydrogen atoms).

Next, the reaction liquid (varnish) was applied to a glass substrate having a size of 76 mm in length and 52 mm in width using a spin coater to form a coating film of the varnish on the glass substrate. Then, the glass plate having the coating film formed thereon was dried under reduced pressure at 70° C. for 30 minutes. Next, the glass plate having the coating film formed thereon was set in an inert oven, and nitrogen purging was performed. Next, under a nitrogen stream, the temperature was raised to 135° C. and held for 1 hour, and the temperature was further raised to 350° C. and held for 1 hour, and then a polyimide was formed on the glass substrate by operating the inert oven so as to cool to room temperature, and a glass substrate coated with a film made of polyimide was obtained. Next, the film made of polyimide was peeled off from the glass substrate to obtain a colorless and transparent film made of polyimide (the step of obtaining such a film is hereinafter referred to as the “film preparation step”). Note that it is found that the obtained polyimide forming the film is a polyimide that contains a repeating unit (A) represented by the following general formula (101) derived from the exo/exo type BzDA used:

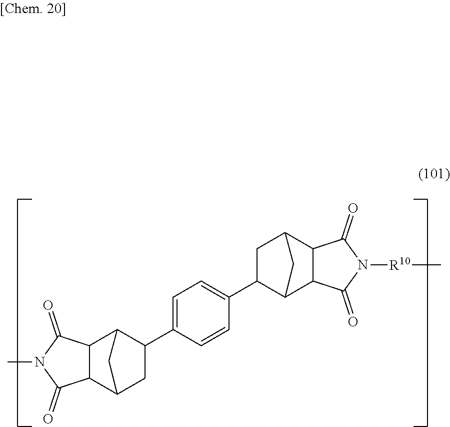

and in which, in the repeating unit (A), the content of the repeating unit having an exo/exo type three-dimensional structure represented by the following general formula (102):

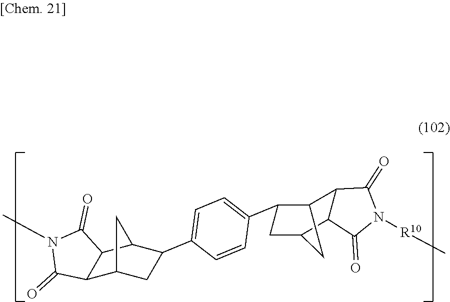

is 100% by mass (each imide ring bonded to the norbornane ring in the formula is a repeating unit taking an exo conformation with respect to the norbornane ring to be bonded) (note that R¹⁰s in the formulas (101) and (102) are each a divalent group obtained by removing two amino groups from DABAN).

### Comparative Example 3

Into a 50 mL flask, 2.70 g (11.9 mmol) of DABAN as an aromatic diamine and 4.88 g (12.0 mmol) of the endo/endo type BzDA obtained in Comparative Example 2 as a tetracarboxylic dianhydride were introduced. Next, into the flask, 10.1 g of dimethylacetamide (N,N-dimethylacetamide) as an organic solvent, 7.6 g of y-butyrolactone as an organic solvent, and 0.061 g (0.50 mmol) of triethylamine as a reaction accelerator were introduced to obtain a mixture liquid. Then, the mixture liquid thus obtained was stirred while heating under a nitrogen atmosphere under a temperature condition of 180° C. for 6 hours to obtain a viscous and uniform pale yellow reaction liquid (varnish). Next, the varnish was applied to a glass substrate having a size of 76 mm in length and 52 mm in width using a spin coater to form a coating film of the varnish on the glass substrate. Then, the glass substrate having the coating film formed thereon was set in an inert oven, and nitrogen purging was performed. Next, in the inert oven, under a nitrogen stream, the temperature was raised to 60° C. and held for 4 hours, and the temperature was then raised to 250° C. and held for 1 hour, and then a polyimide was formed on the glass substrate by operating the inert oven so as to cool to room temperature, and a glass substrate coated with a film made of polyimide was obtained. Next, the film made of polyimide was peeled off from the glass substrate to obtain a colorless and transparent film made of polyimide. It is found that the thus-obtained polyimide forming the film is a polyimide that contains a repeating unit (A) represented by the above general formula (101) derived from the endo/endo type BzDA used, and in which, in the repeating unit (A), the content of the repeating unit having an endo/endo type three-dimensional structure represented by the following formula (103):

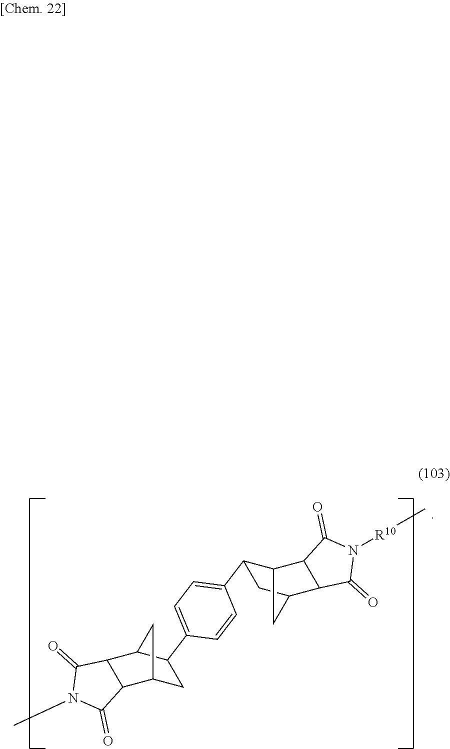

is 100% by mass (each imide ring bonded to the norbornane ring in the formula is a repeating unit taking an endo conformation with respect to the norbornane ring to be bonded) (note that R¹⁰s in the formulas (101) and (103) are each a divalent group obtained by removing two amino groups from DABAN).

### [Evaluation of Characteristics of Polyimides Obtained in Example 3 and Comparative Example 3]

The following measurement methods were employed to subject the polyimides (films) obtained in Example 3 and Comparative Example 3 to the measurement of linear expansion coefficient, glass transition temperature, total luminous transmittance, 5% weight loss temperature (Td5%), HAZE, and YI (note that the polyimides (films) obtained in Examples 4 to 18 and Comparative Examples 4 to 8 described later were also measured by employing the following measurement methods, respectively). Table 1 presents the results obtained together with the film thickness of each film.

### <Method of Measuring Linear Expansion Coefficient (CTE)>

The linear expansion coefficient was measured as follows. Specifically, a film in a size of 20 mm in length and 5 mm in width (the thickness of the sample was the same as the thickness of the film obtained in each of Examples and the like) was cut out from the polyimide (film) obtained in each of Examples and the like. By using this film as a measurement sample, the change in length of the sample was measured from 50° C. to 200° C. under a nitrogen atmosphere in a tensile mode (49 mN) by employing a condition of a rate of temperature rise of 5° C./minute with a thermomechanical analyzer (manufactured by Rigaku Corporation under the trade name of “TMA 8311”) being used as a measuring apparatus. Then, the average value of the changes in length per Celsius degree in the temperature range from 100° C. to 200° C. was determined.

### <Method of Measuring Glass Transition Temperature (Tg)>

The glass transition temperature (unit: ° C.) was measured as follows. Specifically, a film in a size of 20 mm in length and 5 mm in width (the thickness of the sample was the same as the thickness of the film obtained in each of Examples and the like) was cut out from the polyimide (film) obtained in each of Examples and the like. By using this film as a measurement sample, the TMA curve was determined by performing measurement under a nitrogen atmosphere in a tensile mode (49 mN) by employing a condition of a rate of temperature rise of 5° C./minute with a thermomechanical analyzer (manufactured by Rigaku Corporation under the trade name of “TMA 8311”) being used as a measuring apparatus. The curves before and after the inflection point of the TMA curve due to the glass transition were extrapolated, thereby determining the value (unit: ° C.) of the glass transition temperature (Tg) of the resin constituting the film obtained in each of Examples and the like.

### <Method of Measuring Total Luminous Transmittance>

The value of the total luminous transmittance (unit: %) was determined as follows. The polyimide (film) obtained in each of Examples and the like was used as it was as a sample for measurement, and the trade name “Haze Meter NDH-5000” manufactured by NIPPON DENSHOKU INDUSTRIES CO., LTD. was used as a measuring apparatus to perform measurement in accordance with JIS K7361-1 (issued in 1997).

### <Measurement of 5% Weight Loss Temperature (Td5%)>

The 5% weight loss temperature (unit: ° C.) was measured as follows using the polyimide film obtained in each of Examples and the like. Specifically, first, 2 to 4 mg of a sample was prepared from the polyimide film obtained in each of Examples, and the sample was placed in an aluminum sample pan. A thermogravimetric analyzer (under the trade name of “TG/DTA7200” manufactured by SII Nanotechnology Inc.) was used as the measuring apparatus. The scanning temperature was set from 40° C. to 200° C. under a nitrogen gas atmosphere, and the sample was heated from room temperature at a heating rate of 10° C./minute and held at 200° C. for 1 hour. The weight at this point was set as the zero point. After that, the scanning temperature was set from 200° C. to 550° C., and heating was performed from 200° C. under the condition of a rate of temperature rise of 10° C./minute to measure the temperature at which the weight of the sample used was reduced by 5%.

### <Method of Measuring HAZE>

The HAZE (turbidity) was determined as follows. The polyimide (film) obtained in each of Examples and the like was used as it was as a sample for measurement, and the trade name “Haze Meter NDH-5000” manufactured by NIPPON DENSHOKU INDUSTRIES CO., LTD. was used as a measuring apparatus to perform measurement in accordance with JIS K7136 (issued in 2000).

### <Measurement of YI>

The yellowness index (YI) was determined as followed. The trade name “Spectrophotometer SD6000” manufactured by NIPPON DENSHOKU INDUSTRIES CO., LTD. was used as a measuring apparatus to perform measurement in accordance with ASTM E313-05 (issued in 2005).

TABLE 1

| | | | Film | | Tg | Total Luminous | | | |
|---|---|---|---|---|---|---|---|---|---|
| | Tetracarboxylic | Aromatic | Thickness | CTE | (TMA) | Transmittance | Td5% | HAZE | |
| | Dianhydride | Diamine | (μm) | (ppm/K) | (° C.) | (%) | (° C.) | (%) | YI |
| Example 3 | Exo/Exo Type | DABAN | 12 | 34 | 392 | 87 | 460 | 0.96 | 3.4 |
| | BzDA | | | | | | | | |
| Comparative | Endo/Endo Type | DABAN | 30 | 55 | 401 | 89 | 464 | 0.87 | 1.3 |
| Example 3 | BzDA | | | | | | | | |

As is clear from the results presented in Table 1, it was confirmed that the polyimides obtained in Example 3 and Comparative Example 3 both had a total luminous transmittance of 80% or more, and the transparency was at a sufficiently high level. In addition, it was confirmed that the polyimide obtained in Example 3 had a Tg of 449° C., a very high value, and the heat resistance based on Tg was at a very high level. In addition, in the case where the repeating unit of the polyimide was composed of a repeating unit having an exo/exo type three-dimensional structure (Example 3), it was confirmed that the polyimide had a lower linear expansion coefficient as compared with the case where the repeating unit of the polyimide was a repeating unit having an endo/endo type three-dimensional structure (Comparative Example 3).

### Example 4

Under a nitrogen atmosphere, into a 15 mL screw tube, 0.495 g (2.46 mmol) of 4,4′-diaminodiphenyl ether (DDE) was introduced as an aromatic diamine, and also, 1.01 g (2.46 mmol) of the exo/exo type BzDA obtained in Example 2 was introduced as a tetracarboxylic dianhydride. Next, 5.97 g of N,N′-dimethylacetamide (DMAc) as a solvent was added into the screw tube to obtain a mixture liquid. Next, the obtained mixture liquid was stirred under a nitrogen atmosphere and under a temperature condition of room temperature for 2 days to obtain a reaction liquid (varnish) (the step of obtaining such a reaction liquid (varnish) is hereinafter referred to as the “varnish preparation step”). Note that it is found that the varnish contains a polyamic acid in which a repeating unit (I) is contained represented by the general formula (5) derived from the exo/exo type BzDA used, and in which, in the repeating unit (I), the content of the repeating unit having an exo/exo type three-dimensional structure represented by the above general formula (6) is 100% by mass (note that, in the formulas (5) and (6), A is a p-phenylene group, R¹⁰ is a divalent group obtained by removing two amino groups from DDE, and R^{a} and Y are both hydrogen atoms).

Next, the reaction liquid (varnish) was applied to a glass substrate having a size of 76 mm in length and 52 mm in width using a spin coater to form a coating film of the varnish on the glass substrate. Then, the glass plate having the coating film formed thereon was set in an inert oven, and nitrogen purging was performed. Next, in the inert oven, under a nitrogen stream, the temperature was raised to 70° C. and held for 3 hours, the temperature was then raised to 135° C. and held for 1 hour, and the temperature was further raised to 350° C. and held for 1 hour, and then a polyimide was formed on the glass substrate by operating the inert oven so as to cool to room temperature, and a glass substrate coated with a film made of polyimide was obtained. Next, the film made of polyimide was peeled off from the glass substrate to obtain a colorless and transparent film made of polyimide (the step of obtaining such a film is hereinafter referred to as the “film preparation step”). Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that R¹⁰s in the formulas (101) and (102) are each a divalent group obtained by removing two amino groups from DDE).

### Comparative Example 4

A reaction liquid (varnish) was produced in the same manner as in the varnish preparation step employed in Example 4 except that the endo/endo type BzDA obtained in Comparative Example 2 was used as the tetracarboxylic dianhydride instead of the exo/exo type BzDA obtained in Example 2. In addition, a colorless and transparent film made of polyimide was obtained in the same manner as in the film preparation step employed in Example 4 except that the reaction liquid (varnish) thus obtained was used, and the condition for operating the inert oven at the time of forming the polyimide was changed to the condition of “under a nitrogen stream, the temperature is raised to 60° C. and held for 4 hours, and then the temperature is raised to 350° C. and held for 1 hour, and then cooled to room temperature.” Note that it is found that the obtained polyimide forming the film is a polyimide that contains a repeating unit (A) represented by the above general formula (101) derived from the endo/endo type BzDA used, and in which, in the repeating unit (A), the content of the repeating unit having an endo/endo type three-dimensional structure represented by the above general formula (103) is 100% by mass (note that R¹⁰s in the formulas (101) and (103) are each a divalent group obtained by removing two amino groups from DDE).

### [Evaluation of Characteristics of Polyimides Obtained in Example 4 and Comparative Example 4]

The above-described measurement methods were employed to subject the polyimides (films) obtained in Example 4 and Comparative Example 4 to the measurement of linear expansion coefficient, glass transition temperature, total luminous transmittance, 5% weight loss temperature (Td5%), HAZE, and YI. Table 2 presents the results obtained together with the film thickness of each film.

TABLE 2

| | | | Film | | Tg | Total Luminous | | | |
|---|---|---|---|---|---|---|---|---|---|
| | Tetracarboxylic | Aromatic | Thickness | CTE | (TMA) | Transmittance | Td5% | HAZE | |
| | Dianhydride | Diamine | (μm) | (ppm/K) | (° C.) | (%) | (° C.) | (%) | YI |
| Example 4 | Exo/Exo Type | DDE | 18 | 56 | 340 | 89 | 457 | 0.83 | 2.6 |
| | BzDA | | | | | | | | |
| Comparative | Endo/Endo Type | DDE | 15 | 67 | 343 | 90 | 475 | 1.3 | 1.6 |
| Example 4 | BzDA | | | | | | | | |

As is clear from the results presented in Table 2, it was confirmed that the polyimides obtained in Example 4 and Comparative Example 4 both had a total luminous transmittance of 80% or more, and the transparency was at a sufficiently high level. In addition, it was confirmed that the polyimides obtained in Example 4 and Comparative Example 4 had a Tg of 250° C. or higher (as is clear from the description in Table 2, both have a Tg of 340° C. or higher), and the heat resistance based on Tg was at a sufficiently high level for both cases. Moreover, in the case where the repeating unit of the polyimide was composed of a repeating unit having an exo/exo type three-dimensional structure (Example 4), it was confirmed that the polyimide had a lower linear expansion coefficient as compared with the case where the repeating unit of the polyimide was a repeating unit having an endo/endo type three-dimensional structure (Comparative Example 4).

### Example 5

Under a nitrogen atmosphere, into a 15 mL screw tube, 0.719 g (2.46 mmol) of 1,3-bis(4-aminophenoxy)benzene (TPE-R) was introduced as an aromatic diamine, and also, 1.01 g (2.46 mmol) of the exo/exo type BzDA obtained in Example 2 was introduced as a tetracarboxylic dianhydride. Next, 6.90 g of N,N′-dimethylacetamide (DMAc) as a solvent was added into the screw tube to obtain a mixture liquid. Next, the obtained mixture liquid was stirred under a nitrogen atmosphere and under a temperature condition of room temperature for 2 days to obtain a reaction liquid (varnish) (the step of obtaining such a reaction liquid (varnish) is hereinafter referred to as the “varnish preparation step”). Note that it is found that the varnish contains a polyamic acid in which a repeating unit (I) is contained represented by the general formula (5) derived from the exo/exo type BzDA used, and in which, in the repeating unit (I), the content of the repeating unit having an exo/exo type three-dimensional structure represented by the above general formula (6) is 100% by mass (note that, in the formulas (5) and (6), A is a p-phenylene group, R¹⁰ is a divalent group obtained by removing two amino groups from TPE-R, and R^{a} and Y are both hydrogen atoms).

Next, the reaction liquid (varnish) was applied to a glass substrate having a size of 76 mm in length and 52 mm in width using a spin coater to form a coating film of the varnish on the glass substrate. Then, the glass plate having the coating film formed thereon was set in an inert oven, and nitrogen purging was performed. Next, in the inert oven, under a nitrogen stream, the temperature was raised to 70° C. and held for 3 hours, and the temperature was then raised to 300° C. and held for 1 hour, and then a polyimide was formed on the glass substrate by operating the inert oven so as to cool to room temperature, and a glass substrate coated with a film made of polyimide was obtained. Next, the film made of polyimide was peeled off from the glass substrate to obtain a colorless and transparent film made of polyimide (the step of obtaining such a film is hereinafter referred to as the “film preparation step”). Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that R¹⁰s in the formulas (101) and (102) are each a divalent group obtained by removing two amino groups from TPE-R).

### Comparative Example 5

A reaction liquid (varnish) was produced in the same manner as in the varnish preparation step employed in Example 5 except that the endo/endo type BzDA obtained in Comparative Example 2 was used as the tetracarboxylic dianhydride instead of the exo/exo type BzDA obtained in Example 2. In addition, a colorless and transparent film made of polyimide was obtained in the same manner as in the film preparation step employed in Example 5 except that the reaction liquid (varnish) thus obtained was used, and the condition for operating the inert oven at the time of forming the polyimide was changed to the condition of “under a nitrogen stream, the temperature is raised to 60° C. and held for 4 hours, and then the temperature is raised to 350° C. and held for 1 hour, and then cooled to room temperature.” Note that it is found that the obtained polyimide forming the film is a polyimide that contains a repeating unit (A) represented by the above general formula (101) derived from the endo/endo type BzDA used, and in which, in the repeating unit (A), the content of the repeating unit having an endo/endo type three-dimensional structure represented by the above general formula (103) is 100% by mass (note that R¹⁰s in the formulas (101) and (102) are each a divalent group obtained by removing two amino groups from TPE-R).

### [Evaluation of Characteristics of Polyimides Obtained in Example 5 and Comparative Example 5]

The above-described measurement methods were employed to subject the polyimides (films) obtained in Example 5 and Comparative Example 5 to the measurement of linear expansion coefficient, glass transition temperature, total luminous transmittance, 5% weight loss temperature (Td5%), HAZE, and YI. Table 3 presents the results obtained together with the film thickness of each film.

TABLE 3

| | | | Film | | Tg | Total Luminous | | | |
|---|---|---|---|---|---|---|---|---|---|
| | Tetracarboxylic | Aromatic | Thickness | CTE | (TMA) | Transmittance | Td5% | HAZE | |
| | Dianhydride | Diamine | (μm) | (ppm/K) | (° C.) | (%) | (° C.) | (%) | YI |
| Example 5 | Exo/Exo Type | TPE-R | 37 | 66 | 274 | 89 | 457 | 0.60 | 2.5 |
| | BzDA | | | | | | | | |
| Comparative | Endo/Endo Type | TPE-R | 13 | 74 | 274 | 89 | 465 | 1.3 | 1.4 |
| Example 5 | BzDA | | | | | | | | |

As is clear from the results presented in Table 3, it was confirmed that the polyimides obtained in Example 5 and Comparative Example 5 both had a total luminous transmittance of 80% or more, and the transparency was at a sufficiently high level. In addition, it was confirmed that the polyimides obtained in Example 5 and Comparative Example 5 had a Tg of 250° C. or higher, and the heat resistance based on Tg was at a sufficiently high level for both cases. Moreover, in the case where the repeating unit of the polyimide was composed of a repeating unit having an exo/exo type three-dimensional structure (Example 5), it was confirmed that the polyimide had a lower linear expansion coefficient as compared with the case where the repeating unit of the polyimide was a repeating unit having an endo/endo type three-dimensional structure (Comparative Example 5).

### Example 6

A reaction liquid (varnish) was produced in the same manner as in the varnish preparation step used in Example except that 0.788 g (2.46 mmol) of 2,2′-bis(trifluoromethyl)benzidine (TFMB) was used as the aromatic diamine instead of using DABAN, and 4.17 g of N,N′-dimethylacetamide (DMAc) was used as the solvent instead of TMU. In addition, a colorless and transparent film made of polyimide was obtained in the same manner as in the film preparation step employed in Example 3 except that the reaction liquid (varnish) thus obtained was used. Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that R¹⁰s in the formulas (101) and (102) are each a divalent group obtained by removing two amino groups from TFMB). The film thickness of the polyimide (film) obtained in Example 6 was 13 μm. Moreover, the polyimide (film) obtained in Example 6 was subjected to measurement of various characteristics by employing the above-mentioned measuring method, and the linear expansion coefficient (CTE) was 54 ppm/K, the glass transition temperature was 357° C., the total luminous transmittance was 90%, Td5% was 443° C., HAZE was 0.84%, and YI was 3.3.

### Example 7

Into a 50 mL flask, 3.20 g (10.0 mmol) of TFMB as an aromatic diamine and 4.06 g (10.0 mmol) of the exo/exo type BzDA obtained in Example 2 as a tetracarboxylic dianhydride were introduced. Next, into the flask, 14.5 g of N,N-dimethylacetamide (DMAc) as an organic solvent, 14.5 g of γ-butyrolactone as an organic solvent, and 0.051 g (0.509 mmol) of triethylamine as a reaction accelerator were introduced to obtain a mixture liquid. Then, the mixture liquid thus obtained was stirred while heating under a nitrogen atmosphere under a temperature condition of 180° C. for 6 hours to obtain a viscous and uniform pale yellow reaction liquid (varnish). Next, the varnish was applied to a glass substrate having a size of 76 mm in length and 52 mm in width using a spin coater to form a coating film of the varnish on the glass substrate. Then, the glass substrate having the coating film formed thereon was dried at 70° C. for 30 minutes under reduced pressure. Next, the glass substrate having the coating film formed thereon was set in an inert oven, and nitrogen purging was performed. Next, in the inert oven, the temperature was raised to 350° C. and held for 1 hour under a nitrogen stream, and then a polyimide was formed on the glass substrate by operating the inert oven so as to cool to room temperature, and a glass substrate coated with a film made of polyimide was obtained. Next, the film made of polyimide was peeled off from the glass substrate to obtain a colorless and transparent film made of polyimide. Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that R¹⁰s in the formulas (101) and (102) are each a divalent group obtained by removing two amino groups from TFMB).

### Example 8

A colorless and transparent film made of polyimide was obtained in the same manner as in Example 7 except that a mixture of 2.44 g (6.00 mmol) of exo/exo type BzDA obtained in Example 2 and 1.63 g (4.00 mmol) of endo/endo type BzDA obtained in Comparative Example 2 (mixture having an exo/exo type BzDA content of 60% by mass) was used as the tetracarboxylic dianhydride instead of using the exo/exo type BzDA obtained in Example 2 alone, the amount of DMAc used in obtaining the mixture liquid was changed to 5.45 g, the amount of γ-butyrolactone used in obtaining the mixture liquid was changed to 5.45 g, a solution diluted by adding 3.05 g each of DMAc and γ-butyrolactone after completion of the reaction was used as a reaction liquid (varnish) instead of using the solution obtained after completion of the reaction (mixture liquid after the reaction) (after stirring while heating the mixture liquid under a nitrogen atmosphere under a temperature condition of 180° C. for 6 hours) as it was as a reaction liquid (varnish), and the time for holding at 350° C. in the inert oven was changed from 1 hour to 1.5 hours. Note that it is found that the obtained polyimide forming the film is a polyimide that contains a repeating unit (A) represented by the above general formula (101) derived from the tetracarboxylic dianhydride used (content of exo/exo type BzDA: 60% by mass), and in which, in the repeating unit (A), the content of the repeating unit having an exo/exo type three-dimensional structure represented by the above general formula (102) is 60% by mass (note that R¹⁰s in the formulas (101) and (102) are each a divalent group obtained by removing two amino groups from TFMB).

### Comparative Example 6

A colorless and transparent film made of polyimide was obtained in the same manner as in Example 7 except that a mixture of 2.03 g (5.00 mmol) of exo/exo type BzDA obtained in Example 2 and 2.03 g (5.00 mmol) of endo/endo type BzDA obtained in Comparative Example 2 (mixture having an exo/exo type BzDA content of 50% by mass) was used as the tetracarboxylic dianhydride instead of using the exo/exo type BzDA obtained in Example 2 alone, the amount of DMAc used was changed to 8.5 g, and the amount of γ-butyrolactone used was changed to 8.5 g. Note that it is found that the obtained polyimide forming the film is a polyimide that contains a repeating unit (A) represented by the above general formula (101) derived from the tetracarboxylic dianhydride used (content of exo/exo type BzDA: 50% by mass), and in which, in the repeating unit (A), the content of the repeating unit having an exo/exo type three-dimensional structure represented by the above general formula (102) is 50% by mass (note that R¹⁰s in the formulas (101) and (102) are each a divalent group obtained by removing two amino groups from TFMB).

### Comparative Example 7

A colorless and transparent film made of polyimide was obtained in the same manner as in Example 8 except that 8.13 g (20.0 mmol) of endo/endo type BzDA obtained in Comparative Example 2 was used alone as a tetracarboxylic dianhydride instead of using a mixture of the exo/exo type BzDA obtained in Example 2 and the endo/endo type BzDA obtained in Comparative Example 2, the amount of TFMB used was 6.40 (20.0 mmol) g, 7.3 g of N-methylpyrrolidone was used instead of DMAc in obtaining the mixture liquid, the amount of γ-butyrolactone used in obtaining the mixture liquid was changed to 7.3 g, the amount of triethylamine used was changed to 0.202 g (2.00 mmol), 18.7 g of y-butyrolactone was added and diluted after completion of the reaction instead of adding DMAc and γ-butyrolactone and diluting after completion of the reaction (after stirring while heating the mixture liquid under a nitrogen atmosphere under a temperature condition of 180° C. for 6 hours). Note that it is found that the obtained polyimide forming the film is a polyimide that contains a repeating unit (A) represented by the above general formula (101) derived from the tetracarboxylic dianhydride used (content of endo/endo type BzDA: 100% by mass), and in which, in the repeating unit (A), the content of the repeating unit having an endo/endo type three-dimensional structure represented by the above general formula (102) is 100% by mass (note that R¹⁰s in the formulas (101) and (102) are each a divalent group obtained by removing two amino groups from TFMB).

### Evaluation of Characteristics of Polyimides Obtained in Examples 7 and 8 and Comparative Examples 6 and 7

The above-described measurement methods were employed to subject the polyimides (films) obtained in Examples 7 and 8 and Comparative Examples 6 and 7 to the measurement of linear expansion coefficient, glass transition temperature, total luminous transmittance, 5% weight loss temperature (Td5%), HAZE, and YI. Table 4 presents the results obtained together with the film thickness of each film.

TABLE 4

| | | | Film | | Tg | Total Luminous | | | |
|---|---|---|---|---|---|---|---|---|---|
| | Tetracarboxylic | Aromatic | Thickness | CTE | (TMA) | Transmittance | Td5% | HAZE | |
| | Dianhydride | Diamine | (μm) | (ppm/K) | (° C.) | (%) | (° C.) | (%) | YI |
| Example 7 | Exo/Exo Type | TFMB | 28 | 49 | 372 | 91 | 455 | 0.50 | 1.6 |
| | BzDA (100 Mass %) | | | | | | | | |
| Example 8 | Exo/Exo Type | TFMB | 28 | 57 | 366 | 90 | 455 | 0.95 | 2.8 |
| | BzDA (60 Mass %) | | | | | | | | |
| | Endo/Endo Type | | | | | | | | |
| | BzDA (40 Mass %) | | | | | | | | |
| Comparative | Exo/Exo Type | TFMB | 70 | 66 | 358 | 89 | 454 | 0.93 | — |
| Example 6 | BzDA (50 Mass %) | | | | | | | | |
| | Endo/Endo Type | | | | | | | | |
| | BzDA (50 Mass %) | | | | | | | | |
| Comparative | Endo/Endo Type | TFMB | 15 | 73 | 324 | 91 | 475 | 0.49 | 1.5 |
| Example 7 | BzDA (100 Mass %) | | | | | | | | |

As is clear from the results presented in Table 4, it was confirmed that the polyimides obtained in Examples 7 and 8 and Comparative Examples 6 and 7 both had a total luminous transmittance of 80% or more, and the transparency was at a sufficiently high level. In addition, it was confirmed that the polyimides obtained in Examples 7 and 8 and Comparative Examples 6 and 7 had a Tg of 250° C. or higher, and the heat resistance based on Tg was at a sufficiently high level for both cases. Moreover, from the results presented in Table 4, it was confirmed that the polyimides (Examples 7 to 8) containing 60% by mass or more of the repeating unit having an exo/exo type three-dimensional structure was a polyimide having a lower linear expansion coefficient than the polyimides (Comparative Examples 6 to 7) in which the content of the repeating unit having an exo/exo type three-dimensional structure was 50% by mass or less, and it was found that, by containing 60% by mass or more of the repeating unit having an exo/exo type three-dimensional structure, it was possible to lower the value of linear expansion coefficient.

### Example 9

A reaction liquid (varnish) was produced in the same manner as in the varnish preparation step used in Example except that 0.901 g (2.46 mmol) of 2,2-bis(3-amino-4-hydroxyphenyl)-hexafluoropropane (Bis-AP-AF) was used as the aromatic diamine instead of using DABAN, and 4.4 g of DMAc was used as a solvent instead of TMU. In addition, a colorless and transparent film made of polyimide was obtained in the same manner as in the film preparation step employed in Example 3 except that the reaction liquid (varnish) thus obtained was used, and the condition for operating the inert oven at the time of forming the polyimide was changed to the condition of “under a nitrogen stream, the temperature is raised to 300° C. and held for 1 hour, and then cooled to room temperature.” Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that R¹⁰s in the formulas (101) and (102) are each a divalent group obtained by removing two amino groups from Bis-AP-AF).

### Example 10

A colorless and transparent film made of polyimide was obtained in the same manner as in Example 7 except that 1.82 g (4.91 mmol) of Bis-AP-AF was used as the aromatic diamine instead of using TFMB, the amount of exo/exo type BzDA obtained in Example 2 was changed to 2.02 g (4.92 mmol), the amount of DMAc used in obtaining the mixture liquid was changed to 4.4 g, the amount of γ-butyrolactone used in obtaining the mixture liquid was changed to 4.4 g, the amount of tri ethyl amine used as a reaction accelerator was changed to 0.0249 g (0.247 mmol), a solution diluted by adding 12.7 g of DMAc after completion of the reaction was used as a reaction liquid (varnish) instead of using the solution obtained after completion of the reaction (mixture liquid after the reaction) (after stirring while heating the mixture liquid under a nitrogen atmosphere under a temperature condition of 180° C. for 6 hours) as it was as a reaction liquid (varnish), and the condition for operating the inert oven was changed to “under a nitrogen stream, the temperature is raised to 250° C. and held for 1 hour, and then cooled to room temperature.” Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that R¹⁰s in the formulas (101) and (102) are each a divalent group obtained by removing two amino groups from Bis-AP-AF).

### Comparative Example 8

A colorless and transparent film made of polyimide was obtained in the same manner as in Example 10 except that 4.07 g (10.0 mmol) of the endo/endo type BzDA obtained in Comparative Example 2 was used as the tetracarboxylic dianhydride instead of the exo/exo type BzDA obtained in Example 2, the amount of Bis-AP-AF used was changed to 3.66 g (10.0 mmol), the amount of DMAc used in obtaining the mixture liquid was changed to 3.8 g, the amount of γ-butyrolactone used in obtaining the mixture liquid was changed to 3.8 g, the amount of triethylamine used was changed to 0.051 g (0.500 mmol), and the amount of DMAc added after completion of the reaction was changed from 12.7 g to 15.6 g. Note that it is found that the obtained polyimide forming the film is a polyimide that contains a repeating unit (A) represented by the above general formula (101) derived from the endo/endo type BzDA used, and in which, in the repeating unit (A), the content of the repeating unit having an endo/endo type three-dimensional structure represented by the above general formula (103) is 100% by mass (note that R¹⁰s in the formulas (101) and (102) are each a divalent group obtained by removing two amino groups from Bis-AP-AF).

### Evaluation of Characteristics of Polyimides Obtained in Examples 9 and 10 and Comparative Example 8

The above-described measurement methods were employed to subject the polyimides (films) obtained in Examples 9 and 10 and Comparative Example 8 to the measurement of linear expansion coefficient, glass transition temperature, total luminous transmittance, 5% weight loss temperature (Td5%), HAZE, and YI. Table 5 presents the results obtained together with the film thickness of each film.

TABLE 5

| | | | Film | | Tg | Total Luminous | | | |
|---|---|---|---|---|---|---|---|---|---|
| | Tetracarboxylic | Aromatic | Thickness | CTE | (TMA) | Transmittance | Td5% | HAZE | |
| | Dianhydride | Diamine | (μm) | (ppm/K) | (° C.) | (%) | (° C.) | (%) | YI |
| Example 9 | Exo/Exo Type | Bis-AP-AF | 38 | 50 | 336 | 90 | 430 | 2.2 | 1.9 |
| | BzDA | | | | | | | | |
| Example 10 | Exo/Exo Type | Bis-AP-AF | 23 | 41 | 345 | 91 | 418 | 0.64 | 0.8 |
| | BzDA | | | | | | | | |
| Comparative | Endo/Endo Type | Bis-AP-AF | 32 | 55 | 316 | 91 | 414 | 0.64 | 1.5 |
| Example 8 | BzDA | | | | | | | | |

As is clear from the results presented in Table 5, it was confirmed that the polyimides obtained in Examples 9 and 10 and Comparative Example 8 both had a total luminous transmittance of 80% or more, and the transparency was at a sufficiently high level. In addition, it was confirmed that the polyimides obtained in Examples 9 and 10 and Comparative Example 8 had a Tg of 250° C. or higher, and the heat resistance based on Tg was at a sufficiently high level. Moreover, in the case where the repeating unit of the polyimide was composed of a repeating unit having an exo/exo type three-dimensional structure (Examples 9 and 10), it was confirmed that the polyimide had a lower linear expansion coefficient as compared with the case where the repeating unit of the polyimide was a repeating unit having an endo/endo type three-dimensional structure (Comparative Example 8).

### Example 11

A reaction liquid (varnish) was produced in the same manner as in the varnish preparation step used in Example 3 except that a mixture of 0.373 g (1.64 mmol) of DABAN and 0.089 g (0.82 mmol) of p-diaminobenzene (PPD) was used as the aromatic diamine instead of using DABAN alone, the amount of TMU used in obtaining the mixture liquid was changed to 5.7 g, and a solution diluted by adding 2.3 g of TMU after completion of the reaction was used as a reaction liquid (varnish) instead of using the solution obtained after completion of the reaction (mixture liquid after the reaction) (after stirring the mixture liquid under a nitrogen atmosphere under a temperature condition of room temperature for 5 days) as it was as a reaction liquid (varnish). In addition, a colorless and transparent film made of polyimide was obtained in the same manner as in the film preparation step employed in Example 3 except that the reaction liquid (varnish) thus obtained was used. Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that, in all repeating units, 50 mol % of the repeating units are such that R¹⁰ is a divalent group obtained by removing two amino groups from DABAN, and the remaining 50 mol % of the repeating units are such that R¹⁰ is a divalent group obtained by removing two amino groups from PPD).

### Example 12

A reaction liquid (varnish) was produced in the same manner as in the varnish preparation step used in Example 3 except that a mixture of 0.394 g (1.23 mmol) of TFMB and 0.133 g (1.23 mmol) of PPD was used as the aromatic diamine instead of using DABAN alone, the amount of TMU used in obtaining the mixture liquid was changed to 3.6 g, and a solution diluted by adding 5.1 g of TMU after completion of the reaction was used as a reaction liquid (varnish) instead of using the solution obtained after completion of the reaction (mixture liquid after the reaction) (after stirring the mixture liquid under a nitrogen atmosphere under a temperature condition of room temperature for 5 days) as it was as a reaction liquid (varnish). In addition, a colorless and transparent film made of polyimide was obtained in the same manner as in the film preparation step employed in Example 11 except that the reaction liquid (varnish) thus obtained was used. Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that, in all repeating units, 50 mol % of the repeating units are such that R¹⁰ is a divalent group obtained by removing two amino groups from TFMB, and the remaining 50 mol % of the repeating units are such that R¹⁰ is a divalent group obtained by removing two amino groups from PPD).

### Example 13

A reaction liquid (varnish) was produced in the same manner as in the varnish preparation step used in Example except that 0.858 g (2.46 mmol) of bis(4-aminophenyl)ester of terephthalic acid (BPTP) was used as the aromatic diamine instead of using DABAN, 5.96 g of N-methylpyrrolidone (NMP) was used as a solvent instead of TMU, and a solution diluted by adding 4.96 g of NMP after completion of the reaction was used as a reaction liquid (varnish) instead of using the solution obtained after completion of the reaction (mixture liquid after the reaction) (after stirring the mixture liquid under a nitrogen atmosphere under a temperature condition of room temperature for 5 days) as it was as a reaction liquid (varnish). In addition, a colorless and transparent film made of polyimide was obtained in the same manner as in the film preparation step employed in Example 11 except that the reaction liquid (varnish) thus obtained was used, and the condition for operating the inert oven at the time of forming the polyimide was changed to the condition of “under a nitrogen stream, the temperature is raised to 135° C. and held for 30 minutes, and then the temperature is raised to 300° C. and held for 1 hour, and then cooled to room temperature.” Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that R¹⁰s in the formulas (101) and (102) are each a divalent group obtained by removing two amino groups from BPTP).

### Example 14

A colorless and transparent film made of polyimide was obtained in the same manner as in Example 10 except that 2.16 g (5.00 mmol) of bis[4-(3-aminophenoxy)phenyl]sulfone (BAPS-M) was used as the aromatic diamine instead of using Bis-AP-AF, the amount of exo/exo type BzDA obtained in Example 2 was changed to 2.03 g (5.00), the amount of DMAc used to obtain the mixture liquid was changed to 8.4 g, the amount of γ-butyrolactone used in obtaining the mixture liquid was changed to 8.4 g, the amount of triethylamine used as a reaction accelerator was changed to 0.0253 g (0.250 mmol), and the solution obtained after completion of the reaction was used as it was as a reaction liquid (varnish) without adding DMAc (without diluting with DMAc) after completion of the reaction (after stirring while heating the mixture liquid under a nitrogen atmosphere under a temperature condition of 180° C. for 6 hours). Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that R¹⁰ s in the formulas (101) and (102) are each a divalent group obtained by removing two amino groups from BAPS-M).

### Example 15

A colorless and transparent film made of polyimide was obtained in the same manner as in Example 10 except that 1.46 g (5.00 mmol) of 1,3-bis(3-aminophenoxy)benzene (APB-N) was used as the aromatic diamine instead of using Bis-AP-AF, the amount of DMAc used in obtaining the mixture liquid was changed to 5.2 g, the amount of γ-butyrolactone used in obtaining the mixture liquid was changed to 5.2 g, and the solution obtained after completion of the reaction was used as it was as a reaction liquid (varnish) without adding DMAc (without diluting with DMAc) after completion of the reaction (after stirring while heating the mixture liquid under a nitrogen atmosphere under a temperature condition of 180° C. for 6 hours). Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that R¹⁰s in the formulas (101) and (102) are each a divalent group obtained by removing two amino groups from APB-N).

### Example 16

A colorless and transparent film made of polyimide was obtained in the same manner as in Example 10 except that 1.01 g (5.14 mmol) of 3,4′-diaminodiphenyl ether (3,4-DDE) was used as the aromatic diamine instead of using Bis-AP-AF, the amount of exo/exo type BzDA obtained in Example 2 was changed to 2.09 g (5.14 mmol), 6.0 g of NMP was used in obtaining the mixture liquid instead of DMAc, the amount of γ-butyrolactone used in obtaining the mixture liquid was changed to 6.0 g, and the solution obtained after completion of the reaction was used as it was as a reaction liquid (varnish) without adding DMAc (without diluting with DMAc) after completion of the reaction (after stirring while heating the mixture liquid under a nitrogen atmosphere under a temperature condition of 180° C. for 6 hours). Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that R¹⁰s in the formulas (101) and (102) are each a divalent group obtained by removing two amino groups from 3,4-DDE).

### Example 17

A colorless and transparent film made of polyimide was obtained in the same manner as in Example 10 except that 1.29 g (5.00 mmol) of 2,2-bis(3-amino-4-hydroxyphenyl)propane (BAPA) was used as the aromatic diamine instead of using Bis-AP-AF, the amount of DMAc used in obtaining the mixture liquid was changed to 6.65 g, the amount of γ-butyrolactone used in obtaining the mixture liquid was changed to 6.65 g, and the amount of DMAc added after completion of the reaction (after stirring while heating the mixture liquid under a nitrogen atmosphere under a temperature condition of 180° C. for 6 hours) was changed from 12.7 g to 5.5 g. Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that R¹⁰s in the formulas (101) and (102) are each a divalent group obtained by removing two amino groups from BAPA).

### Example 18

A colorless and transparent film made of polyimide was obtained in the same manner as in Example 10 except that 1.41 g (5.00 mmol) of 2,2-bis(3-amino-4-hydroxyphenyl)sulfone (BPS-DA) was used as the aromatic diamine instead of using Bis-AP-AF, the amount of the exo/exo type BzDA obtained in Example 2 was 2.03 g (5.00 mmol), and a silicon wafer was used instead of the glass substrate. Note that it is found that the obtained polyimide forming the film is a polyimide that contains a repeating unit (A) represented by the above general formula (101) derived from the exo/exo type BzDA used, and in which, in the repeating unit (A), the content of the repeating unit having an exo/exo type three-dimensional structure represented by the above general formula (102) is 100% by mass (note that R¹⁰s in the formulas (101) and (102) are each a divalent group obtained by removing two amino groups from BPS-DA).

### Evaluation of Characteristics of Polyimides Obtained in Examples 11 to 18

The above-described measurement methods were employed to subject the polyimides (films) obtained in Examples 11 to 18 to the measurement of linear expansion coefficient, glass transition temperature, total luminous transmittance, 5% weight loss temperature (Td5%), HAZE, and YI. Table 6 presents the results obtained together with the film thickness of each film.

TABLE 6

| | | | Film | | Tg | Total Luminous | | | |
|---|---|---|---|---|---|---|---|---|---|
| | Tetracarboxylic | Aromatic | Thickness | CTE | (TMA) | Transmittance | Td5% | HAZE | |
| | Dianhydride | Diamine | (μm) | (ppm/K) | (° C.) | (%) | (° C.) | (%) | YI |
| Example 11 | Exo/Exo Type | DABAN (50 mol %) | 15 | 37 | 387 | 87 | 452 | 1.1 | 4.0 |
| | BzDA | PPD (50 mol %) | | | | | | | |
| Example 12 | Exo/Exo Type | TFMB (50 mol %) | 20 | 54 | 364 | 90 | 452 | 0.79 | 2.9 |
| | BzDA | PPD (50 mol %) | | | | | | | |
| Example 13 | Exo/Exo Type | BPTP | 15 | 21 | 431 | 86 | 438 | 1.1 | — |
| | BzDA | | | | | | | | |
| Example 14 | Exo/Exo Type | BAPS-M | 20 | 55 | 257 | 89 | 461 | 0.85 | 0.8 |
| | BzDA | | | | | | | | |
| Example 15 | Exo/Exo Type | APB-N | 30 | 58 | — | 89 | 458 | 1.30 | 1.1 |
| | BzDA | | | | | | | | |
| Example 16 | Exo/Exo Type | 3,4-DDE | 22 | 52 | 296 | 88 | 453 | 0.74 | 3.4 |
| | BzDA | | | | | | | | |
| Example 17 | Exo/Exo Type | BAPA | 17 | 41 | 329 | 89 | — | 0.78 | 1.9 |
| | BzDA | | | | | | | | |
| Example 18 | Exo/Exo Type | BPS-DA | 16 | 41 | 363 | 86 | — | 0.96 | — |
| | BzDA | | | | | | | | |

### INDUSTRIAL APPLICABILITY

As described above, the present invention makes it possible to provide a tetracarboxylic dianhydride that can be used as a raw material monomer for producing a polyimide having a lower linear expansion coefficient while having a sufficiently high level of light transmittance and heat resistance; a carbonyl compound that can be used as a raw material for efficiently producing the tetracarboxylic dianhydride and can be obtained as an intermediate during the production of the tetracarboxylic dianhydride; a polyimide precursor resin that can be suitably used for producing the polyimide having a lower linear expansion coefficient while having a sufficiently high level of light transmittance and heat resistance and can be efficiently produced by using the tetracarboxylic dianhydride; and a polyimide that can have a lower linear expansion coefficient while having a sufficiently high level of light transmittance and heat resistance. Therefore, the tetracarboxylic dianhydride of the present invention is useful as a monomer or the like for producing a polyimide for glass replacement. In addition, the tetracarboxylic dianhydride of the present invention can have sufficiently high solvent solubility, and is also useful as a compound or the like for use in applications such as an epoxy curing agent.

1. A tetracarboxylic dianhydride which is a compound represented by the following general formula (1): 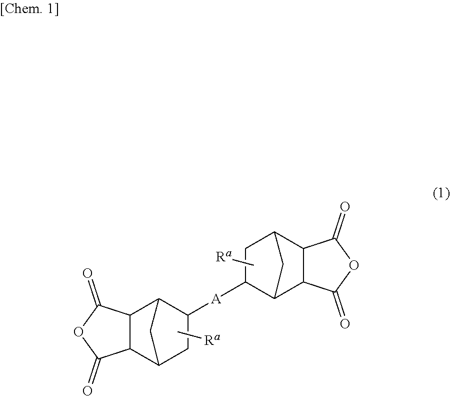
[in the formula (1), A represents one selected from the group consisting of optionally substituted divalent aromatic groups in each of which the number of carbon atoms forming an aromatic ring is 6 to 30, and R^{a}s each independently represent one selected from the group consisting of a hydrogen atom and alkyl groups having 1 to 10 carbon atoms], wherein 60% by mass or more of a stereoisomer contained in the compound is an exo/exo type stereoisomer represented by the following general formula (2): 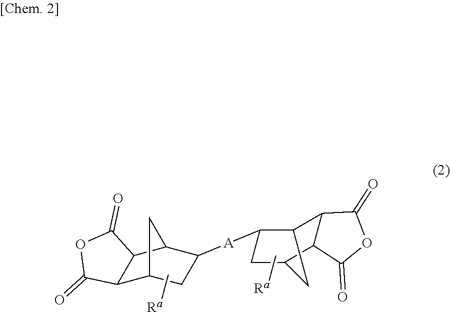
[A and R^{a} in the formula (2) have the same definitions as A and R^{a} in the above general formula (1)].

2. A carbonyl compound which is a compound represented by the following general formula (3): 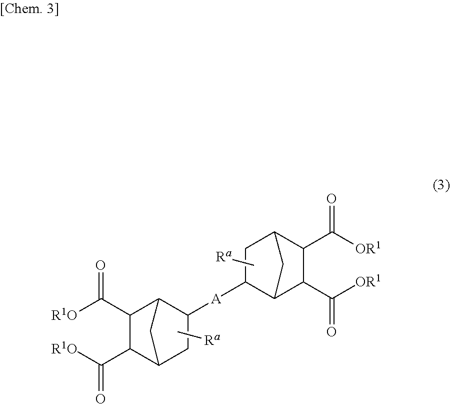
[in the formula (3), A represents one selected from the group consisting of optionally substituted divalent aromatic groups in each of which the number of carbon atoms forming an aromatic ring is 6 to 30, R^{a}s each independently represent one selected from the group consisting of a hydrogen atom and alkyl groups having 1 to 10 carbon atoms, and R¹s each independently represent one selected from the group consisting of a hydrogen atom, alkyl groups having 1 to 10 carbon atoms, cycloalkyl groups having 3 to 10 carbon atoms, alkenyl groups having 2 to 10 carbon atoms, aryl groups having 6 to 20 carbon atoms, and aralkyl groups having 7 to 20 carbon atoms], wherein 60% by mass or more of a stereoisomer contained in the compound is an exo/exo type stereoisomer represented by the following general formula (4): 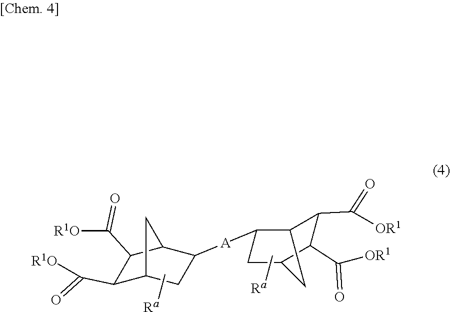
[A, R^{a}, and R¹ in the formula (4) have the same definitions as A, R^{a}, and R¹ in the above general formula (3), respectively].

3. A polyimide precursor resin comprising a repeating unit (I) represented by the following general formula (5): 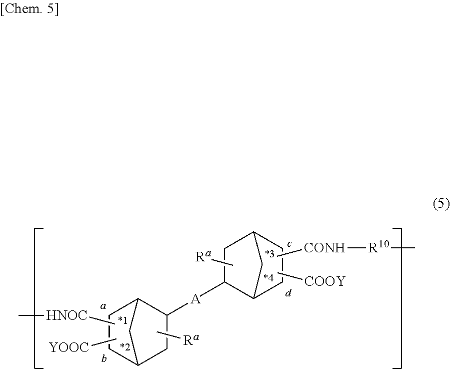
[in the formula (5), A represents one selected from the group consisting of optionally substituted divalent aromatic groups in each of which the number of carbon atoms forming an aromatic ring is 6 to 30, R^{a}s each independently represent one selected from the group consisting of a hydrogen atom and alkyl groups having 1 to 10 carbon atoms, R¹⁰ represents an arylene group having 6 to 50 carbon atoms, Ys each independently represent one selected from the group consisting of a hydrogen atom, alkyl groups having 1 to 6 carbon atoms, and alkylsilyl groups having 3 to 9 carbon atoms, one of the bonder represented by *1 and the bonder represented by *2 is bonded to the carbon atom a forming the norbornane ring, the other of the bonder represented by *1 and the bonder represented by *2 is bonded to the carbon atom b forming the norbornane ring, one of the bonder represented by *3 and the bonder represented by *4 is bonded to the carbon atom c forming the norbornane ring, and the other of the bonder represented by *3 and the bonder represented by *4 is bonded to the carbon atom d forming the norbornane ring], wherein
60% by mass or more of the repeating unit (I) contained in the polyimide precursor resin is a repeating unit having an exo/exo type three-dimensional structure represented by the following general formula (6): 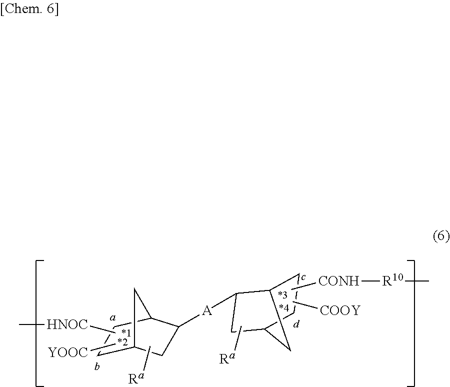
[in the formula (6), A, R^{a}, R¹⁰, and Y have the same definitions as A, R^{a}, R¹⁰, and Y in the general formula (5), respectively, one of the bonder represented by *1 and the bonder represented by *2 is bonded to the carbon atom a forming the norbornane ring, the other of the bonder represented by *1 and the bonder represented by *2 is bonded to the carbon atom b forming the norbornane ring, one of the bonder represented by *3 and the bonder represented by *4 is bonded to the carbon atom c forming the norbornane ring, the other of the bonder represented by *3 and the bonder represented by *4 is bonded to the carbon atom d forming the norbornane ring, and the bonders represented by *1 to *4 have an exo conformation with respect to the norbornane ring to be bonded].

4. A polyimide comprising a repeating unit (A) represented by the following general formula (7): 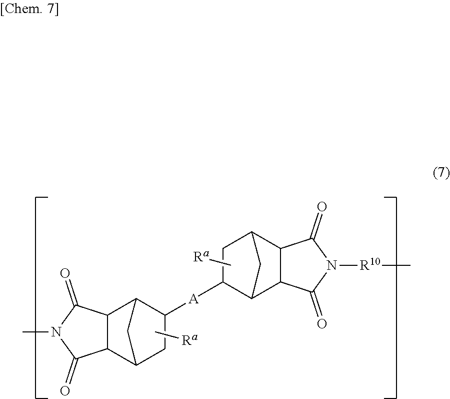
[in the formula (7), A represents one selected from the group consisting of optionally substituted divalent aromatic groups in each of which the number of carbon atoms forming an aromatic ring is 6 to 30, R^{a}s each independently represent one selected from the group consisting of a hydrogen atom and alkyl groups having 1 to 10 carbon atoms, and R¹⁰ represents an arylene group having 6 to 50 carbon atoms], wherein
60% by mass or more of the repeating unit (A) contained in the polyimide is a repeating unit having an exo/exo type three-dimensional structure represented by the following general formula (8): 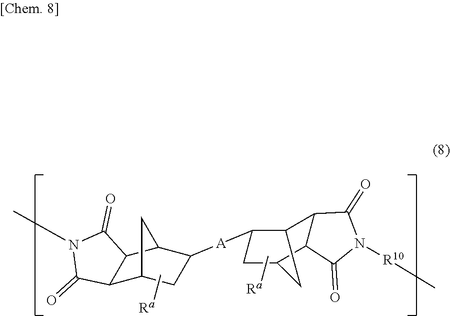
[A, R^{a}, and R¹⁰ in the formula (8) have the same definitions as A, R^{a}, and R¹⁰ in the above general formula (7), respectively].
